(19) United States
(12) Patent Application Publication
(10) Pub. No.: US 2024/0287443 A1
(43) Pub. Date: Aug. 29, 2024

(54) RECOMBINANT ALGAE AND PRODUCTION OF SPIDER SILK PROTEIN FROM THE RECOMBINANT ALGAE

(71) Applicant: Reliance Industries Limited, Mumbai (IN)

(72) Inventors: Manmeet AHUJA, Mumbai (IN); Chitranshu KUMAR, Thane (IN); Anamika UPADHYAY, New Delhi (IN); Vikas Kumar PATEL, Bhadohi (IN); Niraja SONI, Thane (IN); Santanu DASGUPTA, Mumbai (IN); Mansi Pankaj VORA, Mumbai (IN)

(21) Appl. No.: 18/573,085

(22) PCT Filed: Jun. 24, 2022
(86) PCT No.: PCT/IB2022/055878

§ 371 Date: Dec. 21, 2023

(30) Foreign Application Priority Data
Jun. 24, 2021 (IN) 202121012719

(51) Int. Cl. C12N 1/12; C07K 14/435; C12N 9/10; C12R 1/89

(52) U.S. Cl. CPC C12N 1/125; C07K 14/43518; C12N 9/1014; C12R 2001/89; C12Y 201/02001

(57) ABSTRACT

The present disclosure relates to recombinant algae, more particularly to recombinant blue green algae. The disclosure further relates to a method for production of recombinant spider silk protein from the said recombinant algae. The said recombinant algae provides for green technology for the production of spider silk protein. The production of spider silk protein in the said recombinant algae is simple and economical.

### TECHNICAL FIELD

The present disclosure relates to the field of molecular biology. Particularly, the present disclosure relates to recombinant algae, and more particularly to recombinant blue green algae (Cyanobacteria). The disclosure further relates to a method for production of recombinant spider silk protein from the said recombinant algae. The disclosure also relates to the use of recombinant algal cells as described herein for producing recombinant proteins.

### BACKGROUND OF THE DISCLOSURE

Silk polypeptides come from a variety of sources, including bees, moths, spiders, mites and other arthropods. Some organisms make multiple silk fibers with unique sequences, structural elements, and mechanical properties. For example, orb weaving spiders can produce up to seven different types of spider silk that are polymerized into fibers tailored to fit an environmental or lifecycle niche. The fibers are named from the gland they originate and the polypeptides are labelled with the gland abbreviation e.g. Ma for Major Ampullate and Sp for spidroin. In orb weavers, these types include Major Ampullate (MaSp.), Minor Ampullate (MiSP)—together called dragline silk, Flagelliform (Flag), Aciniform (AcSp), Tubuliform (TuSp), and Pyriform (PySP). This combination of polypeptide sequences across fiber types, domains and variation amongst different genus and species of organisms leads to a vast array of potential properties that can be harnessed by commercial production of the recombinant fibers.

Natural spider silk proteins are large (>150 kDa, >1000 amino acids) polypeptides that can be broken down into three domains: a N-terminal non-repetitive domain (NTD), the repeat domain (REP), and the C-terminal non-repetitive domain (CTD). The NTD and CTD are relatively small (150, 100 amino acids respectively) and are believed to confer to the polypeptide aqueous stability, pH sensitivity, and molecular alignment upon aggregation. NTD also has a strongly predicted secretion tag, which is often removed during heterologous expression. The repetitive sequence is rich in glycine and alanine and characterized by stretches of alanine that are interrupted by glycine-rich repeats. The poly alanine regions form hydrophobic crystalline domains that are responsible for the high tensile strength, whereas the glycine-rich regions are hydrophilic and responsible for the links between crystalline domains as well as the elasticity of fiber.

For commercial applications, natural spider silk cannot be conveniently obtained by farming spiders because they are highly territorial and aggressive. This makes it difficult to produce it at commercial scale. Thus, many attempts have been made to produce recombinant silk proteins followed by their spinning into artificial fibers. Recombinant spider silk has been demonstrated from engineered organisms such as bacteria, yeast, mammalian cells and plant systems. One of the biggest challenges currently noted in the efficient production of spider silk proteins is finding the right host for expression of spider silk protein. Secondly, the size of a recombinant spider silk protein is a key factor in controlling the mechanical properties of the spun fiber. Since spider silk proteins contain iterated peptide motifs, they are mainly constructed by using methods such as concatemerization and step-by-step directional approach. Furthermore, mainly plasmid based system used for heterologous expression have a host of drawbacks such as plasmid instability, vector load, reduced cell viability, plasmid loss and increased process cost (antibiotics) during large scale fermentation. Large scale fermentation is expensive which leads to non-economically viable production of spider silk proteins at commercial scale. Further, it was noted that some of the by-products formed during the production of spider silk protein were known to be toxic to the cell and environment. Thus, there was a need for effective and economical means of producing spider silk protein that overcomes the above noted limitations.

### SUMMARY OF THE DISCLOSURE

To meet the growing need or demand for the production of recombinant proteins, particularly silk protein, the Applicant in the present disclosure describes a recombinant algae, particularly recombinant blue green algae capable of producing silk protein including but not limited to spider silk protein. The recombinant algae is capable of producing spider silk protein in effective and economical manner. The recombinant algae does not lead to formation of toxic by-products while producing spider silk protein.

In the present disclosure, it is demonstrated that recombinant spider silk protein made from engineered algae (recombinant algae) described herein provides for an opportunity to harness the technological capabilities and sustainable route to produce advanced biomaterials.

In some embodiments, the present disclosure relates to a recombinant algae capable of expressing gene that code for spider silk protein including but not limited to Major ampullate Spidroin-1 (MaSp1), Major Ampullate Spidroin-2 (MaSp2), Minor Ampullate Spidroin-1 (MiSp1), Minor Ampullate Spidroin-2 (MiSp2), Flagelliform (Flag).

In some embodiments, the present disclosure further relates to a method of producing the recombinant algae capable of expressing the spider silk protein.

In some embodiments, the present disclosure further relates to use of the recombinant algae for producing spider silk protein including but not limited to MaSp1, MaSp2, MiSp1, MiSp2 and flag.

In some embodiments, the present disclosure further relates to a method of producing recombinant spider silk protein by the recombinant algae.

In some embodiments, the present disclosure further relates to method of using the recombinant spider silk protein produced by the recombinant algae for industrial applications.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with detailed description below, are incorporated in and form part of the specification, and serve to further illustrate the embodiments and explain various principles and advantages, in accordance with the present disclosure where:

FIG. 1: illustrates synthetic amino acid sequence of MaSp1 monomer.

FIG. 2: illustrates two glycyl-tRNAs identified in the genome of C.aponinum strain.

FIG. 3: a) illustrates amplification of vector backbone, two glycyl-tRNAs and a GlyASHMT gene where Lane M=DNA Marker, Lane 1=Vector backbone corresponding to Seq ID No. 3, Lane 2=Amplification of glycyl-tRNA 1 corresponding to Seq ID No. 9, Lane 3=Amplification of glycyl-tRNA 2 corresponding to Seq ID No. 10, Lane 4=Amplification of GlyASHMT gene corresponding to Seq ID No. 11: b) illustrates diagnostic PCR confirmation of putative transformants where Lane M=DNA Marker, Lane 1=Colony PCR: c) illustrates isolation of plasmids where Lane M=DNA Marker, Lane 1-3=Purified Plasmid in triplicate; and d) illustrates restriction digestion using NcoI and BamHI single and double digestion for confirmation of putative transformants in E. coli where Lane M=DNA Marker, Lane 1a, 2a,=Digestion of transformed plasmid with NcoI, Lane 3a=Digestion of control plasmid with NcoI, Lane 1b, 2b, =Digestion of transformed plasmid with BamHI, Lane 3b=Digestion of control plasmid with BamHI, Lane 1c, 2c, =Digestion of transformed plasmid with NcoI and BamHI, Lane 3c=Digestion of control plasmid with NcoI, and BamHI (all in triplicate).

FIG. 4: a) illustrates the cyanobacterial transformant with the GlyASHMT cassette grown on BG11 plate with antibiotic; and b) illustrates PCR confirmation of GlyASHMT construct in C.aponinum strain where Lane M=DNA Marker, Lane C=Wild type C.aponinum control, Lanes 1-3=Diagnostic PCR of GlyASHMT integrants.

FIG. 5: a) illustrates release of MaSp1 monomer from commercial vector where Lane M=DNA Marker, Lanes 1-4=release of MaSp1 monomer with BamHI digestion: b) illustrates restriction digestion of putative transformants for monomer MaSp1 in pET30a vector where, Lane C=pET30a empty vector control, Lane 1=pET30a-MaSp1 vector digested with NheI and XhoI enzymes: c) illustrates digestion of pET30a-MaSp1 vector for multimerization of MaSp1 where Lane M=DNA Marker, Lanes 1, 2=pET30-MasSp1 digested with NheI enzyme and Lanes 3,4=pET30-MasSp1 digested with NheI and SpeI enzymes; and d) illustrates restriction digestion of putative transformants for decamer MaSp1 in pET30a vector where Lane M=DNA ladder, Lanes 1-7=putative transformants for decamer (10×) MaSp1-pET30a vector digested with NheI/SpeI enzymes, Lane C1=pentamer (5×) MaSp1-pET30a control vector used for multimerization digested with NheI/SpeI enzymes, Lane C2=Undigested pentamer (5×) MaSp1-pET30a control vector.

FIG. 6: a) illustrates amplification of vector backbone and amplification of decamer MaSp1 from 10 mer pET30a-MaSp1 vector where Lane M=DNA Marker, Lanes 1a-1c=Amplification of backbone corresponding to Seq ID No. 4, Lanes 2a-2c=amplification of decamer MaSp1; and b) illustrates screening of putative transformants in E. coli by restriction digestion where Lane M=DNA Marker and Lane 1=Putative transformant digested with PacI/XhoI.

FIG. 7: a) illustrates WT C.aponinum cells-only control after transformation on antibiotic plate as negative control: b) illustrates the cyanobacterial transformant with the decamer MaSp1 construct in the WT C.aponinum grown on BG11 media plate with antibiotic and c) illustrates the cyanobacterial transformant with the decamer MaSp1 construct in GlyASHMT C.aponinum grown on BG11 media plate with antibiotic.

FIG. 8: illustrates diagnostic colony PCR of a) decamer MaSp1 in WT C.aponinum where Lane M=DNA Marker, Lanes 1-3=PCR amplification of transformants in triplicate to confirm integration at Seq ID No. 22, Lane C=WT C.aponinum control; and b) decamer MaSp1 in GlyASHMT strain where Lane M=DNA Marker, Lanes 1-3-PCR amplification of transformants in triplicate to confirm integration at Seq ID No. 22, Lane C=GlyASHMT C.aponinum control.

FIG. 9: a) illustrates amplification of fragments for cloning of octadecamer (18×) and trigintahexamer (36×) MaSp1 in Seq ID No. 4 where Lane M=DNA Marker, Lane 1=amplification of the octadecamer fragment from MaSp1 18mer-pET30a vector, Lane 2=amplification of the trigintahexamer fragment from MaSp1 36mer-pET30a vector, Lane 3=amplification of backbone corresponding to Seq ID No. 4; and b) illustrates screening of putative transformants of octodecamer MaSp1 in E. coli where Lane M=DNA Marker, Lane C=Undigested plasmid control, Lanes 1-2 restriction digestion of transformants using PacI and XhoI enzymes; and c) illustrates screening of putative transformants of trigintahexamer MaSp1 in E. coli where Lane M=DNA Marker, Lane 1=restriction digestion of transformant using PacI and XhoI enzymes.

FIG. 10: illustrates diagnostic colony PCR of a) octodecamer MaSp1 in WT C.aponinum where Lane M=DNA Marker, Lanes 1-2=PCR amplification of transformants to confirm integration, Lane C=WT C.aponinum control: b) octodecamer MaSp1 in GlyASHMT C.aponinum where Lane M=DNA Marker, Lanes 1-5=PCR amplification of transformants to confirm integration, Lane C=GlyASHMT C.aponinum control: c) trigintahexamer MaSp1 in WT C.aponinum where Lane M=DNA Marker, Lanes 1-3=PCR amplification of transformants to confirm integration, Lane C=WT C.aponinum control; and d) trigintahexamer MaSp1 in GlyASHMT C.aponinum where Lane M=DNA Marker, Lanes 1-3=PCR amplification of transformants to confirm integration, Lane C=GlyASHMT C.aponinum control.

FIG. 11: a) illustrates amplification of fragments for cloning of decamer MaSp1 fragment and backbone for cloning in Seq ID No. 5 where Lane M=DNA Marker, Lane 1=Amplification of decamer fragment from 10mer MaSp1-pET30a vector, Lanes 2-3=amplification of vector backbone corresponding to Seq ID No. 5 in two parts; and b) illustrates screening of putative transformants of decamer MaSp1 in E. coli where Lane M=DNA Marker, Lanes 1-4=by restriction digestion of transformants using SpeI enzyme, Lane C=Empty vector control.

FIG. 12: illustrates putative colonies for a) WT C.aponinum with decamer MaSp1 integrated at Seq ID No. 22: b) GlyASHMT C.aponinum with decamer MaSp1 integrated at Seq ID No. 22: c) GlyASHMT C.aponinum with decamer MaSp1 integrated at Seq ID No. 22 and Seq ID No. 23.

FIG. 13: illustrates diagnostic PCR for a) decamer MaSp1 in WT C.aponinum at Seq ID No. 23 where Lane M=DNA Marker, Lanes 1-5=PCR amplification of transformants to confirm integration, Lane C=WT C.aponinum control: b) decamer MaSp1 in GlyASHMT C.aponinum at Seq ID No. 23 where Lane M=DNA Marker, Lanes 1-4=PCR amplification of transformants to confirm integration, Lane C=GlyASHMT C.aponinum control.

FIG. 14: illustrates diagnostic PCR for a C.aponinum strain containing two copies of decamer MaSp1 at Seq ID No. 22 and Seq ID No. 23 and one copy of GlyASHMT at Seq ID No. 24, wherein a) illustrates diagnostic PCR for MaSp1 at Seq ID No. 23 where Lane M=DNA Marker, Lanes 1-8=PCR amplification of transformants to confirm integration at Seq ID No. 23, Lane C=GlyASHMT C.aponinum control: b) illustrates diagnostic PCR of MaSp1 at Seq ID No. 22 where Lane M=DNA Marker, Lanes 1-4 PCR amplification of transformants to confirm integration at Seq ID No. 22, Lane C1=WT C.aponinum control, Lane C2=GlyASHMT C.aponinum control; and C) illustrates diagnostic PCR of GlyASHMT at Seq ID No. 24 where Lane M=DNA Marker, Lanes 1-4 PCR amplification of transformants to confirm integration at Seq ID No. 24, Lane C1=WT C.aponinum control, Lane C2=GlyASHMT C.aponinum control

FIG. 15: illustrates Western blot analysis of strain expressing the decamer MaSp1 in WT C.aponinum (Lane 1) versus the decamer MaSp1 in GlyASHMT C.aponinum (Lanes 2-6).

FIG. 16: illustrates Western blot analysis of a strain expressing two copies of the decamer MaSp1 in GlyASHMT C.aponinum (Lanes 1, 2 and 3) versus one copy of the decamer MaSp1 in WT C.aponinum (Lanes 4 and 5).

FIG. 17: illustrates western blot analysis of the strain expressing one copy of the octodecamer MaSP1 in GlyASHMT C. aponinum, wherein Lane M=Protein Marker, Lanes 1-5=western blots of putative transformants, Lane C=western blot of decamer MaSP1 in GlyASHMT C. aponinum.

FIG. 18: illustrates Western blot analysis of a strain expressing one copy of the trigintahexamer MaSp1 in GlyASHMT C.aponinum, where Lane M=Protein Marker, Lanes 1-5=western blots of putative transformants, Lane C=western blot of decamer MaSp1 in GlyASHMT C.aponinum.

FIG. 19 a) illustrates amplification of three fragments: Seq ID No. 21 and Seq ID No. 25, amplification of decamer MaSp1 from 10 mer pET30a-MaSp1 vector and amplification of backbone corresponding to Seq ID No. 4, where Lane M=DNA Marker, Lanes 1=amplification of Seq ID No. 21 and Seq ID No. 25, Lane 2=amplification of decamer MaSp1 from 10 mer pET30a-MaSp1 vector, Lane 3=amplification of backbone corresponding to Seq ID No. 4. b) illustrates screening of putative transformants in E. coli by restriction digestion where Lane M=DNA Marker and Lane 1,2,3=Putative transformant digested with PacI/XhoI.

FIG. 20 a) illustrates the cyanobacterial transformant with the decamer MaSp1 construct with Seq ID No. 21 and Seq ID No. 25 in the WT C.aponinum and GlyASHMT C.aponinum grown on BG11 media plate with antibiotic; and b) illustrates diagnostic colony PCR of the cyanobacterial transformants with the decamer MaSp1 construct with Seq ID No. 21 and Seq ID No. 25 in WT C.aponinum and GlyASHMT C.aponinum where Lane M=DNA Marker, Lane 1=confirm integration at Seq ID No. 22 in C.aponinum. Lane 2=confirm integration at Seq ID No. 22 in GlyASHMT C.aponinum. Lane 3=WT C.aponinum control.

### DETAILED DESCRIPTION OF THE DISCLOSURE

### Definitions

Unless otherwise defined, all terms used in the disclosure, including technical and scientific terms, have meaning as commonly understood by one of ordinary skill in the art to which this invention belongs. By means of further guidance, term definitions are included to better understand the present disclosure.

As used herein, the singular forms ‘a’, ‘an’, and ‘the’ include both singular and plural referents unless the context clearly dictates otherwise.

The term ‘comprising’, ‘comprises’ or ‘comprised of as used herein are synonymous with “including’, ‘includes’, ‘containing’ or ‘contains’ and are inclusive or open-ended and do not exclude additional, non-recited members, elements or method steps.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +10% or less, preferably +5% or less, more preferably #1% or less and still more preferably +0.1% or less of and from the specified value, insofar such variations are appropriate to perform the present disclosure. It is to be understood that the value to which the modifier ‘about’ refers is itself also specifically, and preferably disclosed.

The term ‘synthetic’ means to form by using, at least in part, a non-naturally occurring component, composition or biological process. By way of example, ‘synthetic nucleic acid sequence’ ‘synthetic nucleotide sequence’ and/or ‘synthetic polypeptide sequence’ refers to nucleotide sequence, nucleic acid sequence or polypeptide sequence that are prepared by artificial synthesis process as opposed to synthesis in living organism. The term ‘synthetic spider silk protein’ used herein refers to protein produced using non-naturally occurring nucleotide sequences, genetically engineered cells, or methods guided by the hand of man.

The term ‘polynucleotide’ or ‘nucleotide sequence’ or ‘nucleic acid molecule’ is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as DNA/RNA hybrid.

The terms ‘recombinant nucleic acid’ or ‘recombinant nucleic acid molecule’ as used herein generally refer to nucleic acid molecules (such as, e.g., DNA, cDNA or RNA molecules) comprising segments generated and/or joined together using recombinant DNA technology, such as for example molecular cloning and nucleic acid amplification. Usually, a recombinant nucleic acid molecule may comprise one or more non-naturally occurring sequences, and/or may comprise segments corresponding to naturally occurring sequences that are not positioned as they would be positioned in a source genome which has not been modified. When a recombinant nucleic acid molecule replicates in the host organism (herein an algal cell) into which it has been introduced, the progeny nucleic acid molecule(s) are also encompassed within the term “recombinant nucleic acid molecule”. The recombinant nucleic acid molecule can be stably integrated into the genome of a host organism (herein an algal cell), such as for example integrated at one or more random positions or integrated in a targeted manner, such as, e.g., by means of homologous recombination, or the recombinant nucleic acid molecule can be present as or comprised within an extra-chromosomal element, wherein the latter may be auto-replicating.

The term ‘recombinant protein’ or ‘recombinant polypeptide’ or ‘protein of interest’ or ‘polypeptide of interest’ as used herein refers to a polypeptide or protein produced by a host organism (herein an algal cell) through the expression of a recombinant nucleic acid molecule, which has been introduced into said host organism and which comprises a sequence encoding said polypeptide or protein.

The terms ‘recombinant’ or ‘transformed’ as used herein with reference to algal cells/algae, encompass such algal cells into which a recombinant nucleic acid molecule has been introduced, as well as the recombinant progeny of such cells. Hence, the term ‘transformation’ encompasses the introduction or transfer of a foreign nucleic acid such as a recombinant nucleic acid into an algal host cell as defined herein. The so-introduced nucleic acid may be preferably maintained throughout the further growth and cell division of said host cell. The algal cell used in the present disclosure includes but is not limited to Chlamydomonas, Nanochloropsis, Chlorella, Cyanobacteria sp.

The term ‘recombinant algae’ as used herein means photosynthetic microscopic organism, which are transformed with a heterologous gene for expression. For e.g., gene encoding spider silk protein is transformed into an algae, to generate the said ‘recombinant algae’ herein. The said photosynthetic microscopic organism comprises chlorophyll and are capable of obtaining their energy through photosynthesis. The said photosynthetic microscopic organism can be a prokaryotic or eukaryotic organism and includes but is not limited to Chlamydomonas, Nanochloropsis Chlorella, Cyanobacteria aponinum, Synechococcus, Synechosystis, Arthospira. Thus, a recombinant algae within the purview of the present disclosure includes a unicellular or multicellular photosynthetic eukaryotic organism or a unicellular or multicellular photosynthetic prokaryotic organism, such as a blue green algae or cyanobacteria.

The term ‘promoter’ used herein includes transcriptional regulatory sequences required for accurate transcription initiation and where applicable accurate spatial and/or temporal control of gene expression or its response to, e.g., internal or external (e.g., exogenous) stimuli. More particularly, ‘promoter’ may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. The promoters contemplated herein may be constitutive or inducible.

The term ‘construct’ or ‘expression construct’ used herein mean any recombinant nucleic acid molecule such as an expression cassette, plasmid, cosmid, virus, autonomously replicating polynucleotide molecule, phage, or linear or circular, single-stranded or double-stranded, DNA or RNA polynucleotide molecule, derived from any source, capable of genomic integration or autonomous replication, comprising a nucleic acid molecule where one or more nucleic acid sequences has been linked in a functionally operative manner, i.e. operably linked.

In some embodiments, the present disclosure relates to a recombinant algae.

In some embodiments of the present disclosure, the recombinant algae is selected from but not limited to recombinant Chlamydomonas, recombinant Nanochloropsis, recombinant Chlorella and recombinant Cyanobacteria aponinum.

In some embodiments of the present disclosure, the recombinant algae is recombinant Cyanobacteria aponinum.

In some embodiments of the present disclosure, the recombinant algae is engineered to produce protein from spidroin gene, commonly known as spider silk protein.

In some embodiments of the present disclosure, the recombinant algae is engineered to produce spider silk protein including but not limited to Major ampullate Spidroin-1(MaSp1), Major Ampullate Spidroin-2 (MaSp2), Minor Ampullate Spidroin-1 (MiSp1), Minor Ampullate Spidroin-2 (MiSp2), Flagelliform (Flag) and proteins that have at least 80% amino acid sequence similarity to the said MaSp1, MaSp2, MiSp1, MiSp2 and Flag proteins.

In some embodiments of the present disclosure, the recombinant algae is engineered in a manner that there is an improved amino acid flux to aid in expression of the heterologous gene, particularly stable expression of the gene that corresponds to spider silk protein. Thus, while expression of the gene encoding the spider silk proteins herein is responsible for the production of the recombinant spider silk protein by the recombinant algae, this production is stabilized by improved said amino acid flux.

In some embodiments of the present disclosure, the recombinant algae comprises synthetic nucleotide sequence that encodes tRNA specifying a particular amino acid, encoded by a nucleotide sequence selected from a group comprising sequences set forth as SEQ ID No. 9, SEQ ID No. 10 and a combination thereof or nucleotide sequence having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97%, at least 98% or at least 99%, similarity to the sequences selected from a group comprising sequences set forth as SEQ ID No. 9 and SEQ ID No. 10.

In some embodiments of the present disclosure, the recombinant algae comprises at least two synthetic nucleotide sequences that encodes tRNA specifying a particular amino acid.

In some embodiments of the present disclosure, the recombinant algae comprises at least two synthetic nucleotide sequence that encodes tRNA specifying glycine amino acid.

In some embodiments of the present disclosure, the recombinant algae overexpresses Serine Hydroxymethyl Transferase (SHMT) or any other accessory gene for glycine overproduction.

In some embodiments of the present disclosure, the recombinant algae overexpresses SHMT encoded by a nucleotide sequence set forth as SEQ ID No. 11 or nucleotide sequence having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97%, at least 98% or at least 99% similarity to SEQ ID No. 11.

In some embodiments of the present disclosure, the recombinant algae comprises at least one synthetic nucleotide sequence that encodes SHMT. The synthetic nucleotide sequence encoding SHMT in the recombinant algae provides for increased expression of SHMT that contributes to the stable production of spider silk protein.

In some embodiments of the present disclosure, the recombinant algae comprises two synthetic nucleotide sequences that encodes tRNA specifying glycine amino acid and one synthetic nucleotide sequence that encodes serine hydroxymethyl transferase (SHMT). FIG. 3a illustrates amplification of vector backbone of two synthetic nucleic acid sequence that encodes tRNA specifying glycine amino acid (two glycyl-tRNAs) and one synthetic nucleic acid sequence that encodes serine hydroxymethyl transferase (SHMT) (GlyASHMT).

In some embodiments of the present disclosure, the recombinant algae is engineered in a manner that there is an improved flux of glycine within the recombinant algae to aid in expression of the heterologous gene, particularly stable expression of the gene that corresponds to spider silk protein. This strain comprising the improved flux of glycine comprises two synthetic nucleotide sequences encoding tRNA specifying glycine amino acid and one synthetic nucleotide sequence encoding SHMT is referred to as GlyASHMT strain.

In some embodiments of the present disclosure, the recombinant algae comprises at least two synthetic nucleotide sequences that encodes tRNA specifying particular amino acid and at least one synthetic nucleotide sequence that encodes serine hydroxymethyl transferase (SHMT) in addition to nucleotide sequence that encodes tRNA specifying a particular amino acid and nucleic acid sequence that encodes SHMT present in the wild type algae.

In some embodiments of the present disclosure, the recombinant algae comprises at least two synthetic nucleotide sequences that encodes tRNA specifying glycine amino acid and at least one synthetic nucleotide sequence that encodes serine hydroxymethyl transferase (SHMT) in addition to nucleotide sequence that encodes tRNA specifying glycine amino acid and nucleic acid sequence that encodes SHMT present in the wild type algae.

In some embodiments of the present disclosure, the gene encoding the spider silk protein is expressed in the recombinant algae under the influence of promoter selected from a group comprising inducible promoter, constitutive promoter, and a combination thereof.

In some embodiments of the present disclosure, the gene encoding the spider silk protein is expressed in the recombinant algae under the influence of promoter selected from a group of sequences set forth as SEQ ID No. 16, SEQ ID No. 17 SEQ ID No 18, SEQ ID No 19, SEQ ID No. 20 SEQ ID No. 21, SEQ ID No. 25, and any combination thereof.

In some embodiments of the present disclosure, the gene encoding the spider silk protein is expressed in the recombinant algae under the influence of promoter having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97%, at least 98% or at least 99%, similarity to the sequences selected from a group comprising sequences set forth as SEQ ID No. 16, SEQ ID No. 17 SEQ ID No. 18, SEQ ID No. 19, SEQ ID No. 20, SEQ ID No. 21 and SEQ ID No. 25. In some embodiments of the present disclosure, the at least two synthetic nucleic acid sequences that encode tRNA specifying glycine amino acid in the recombinant algae are expressed under the influence of promoters including but not limited to SEQ ID No. 16 and SEQ ID No. 17.

In some embodiments of the present disclosure, the at least one synthetic nucleic acid sequence that encodes SHMT in the recombinant algae is expressed under the influence of promoter including but not limited to SEQ ID No 18.

In some embodiments of the present disclosure, the recombinant algae expresses gene encoding the spider silk protein including but not limited to MaSp1 gene, MaSp2 gene, MiSp1 gene, MiSp2 gene, Flag gene or their variants in one copy (monomer), two copies (dimer), three copies (trimer) or multiple copies (concatemer).

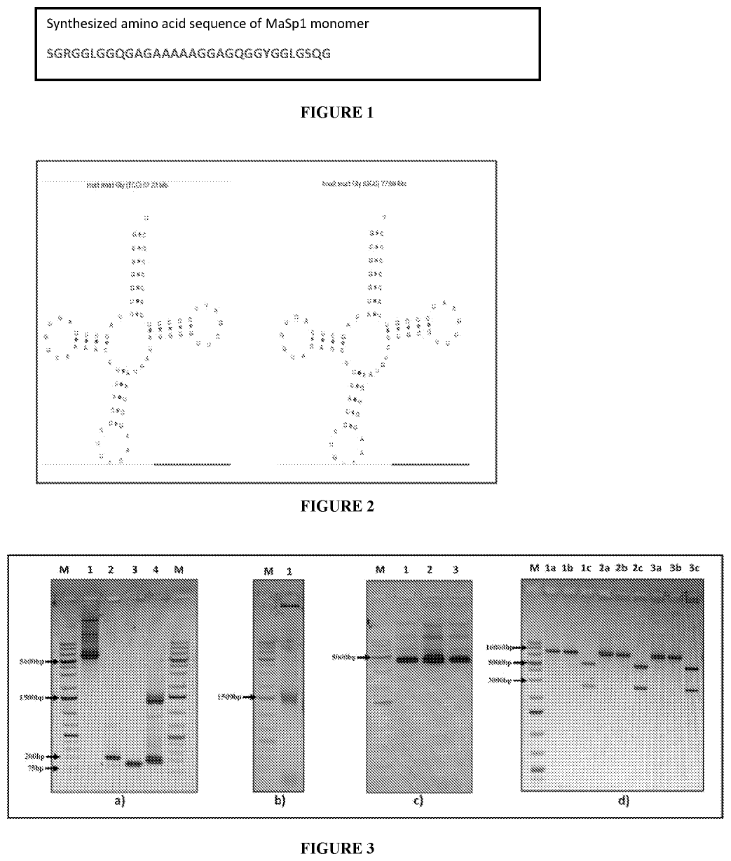

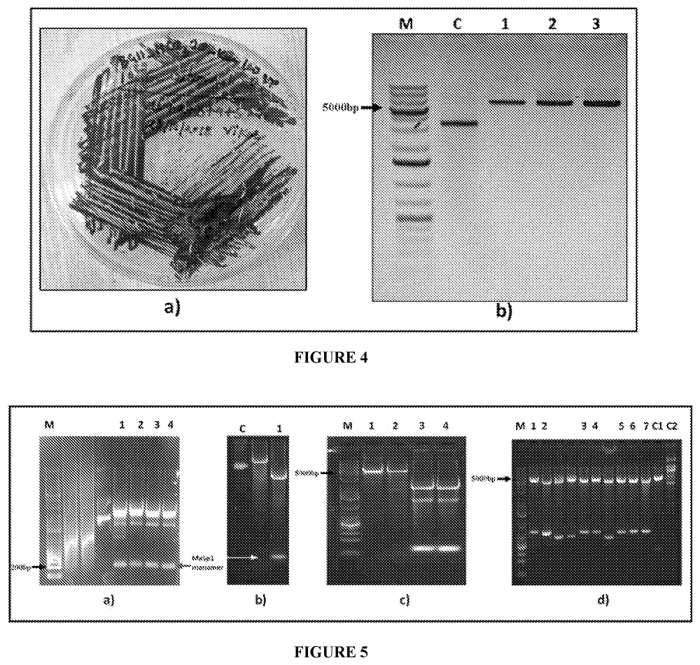

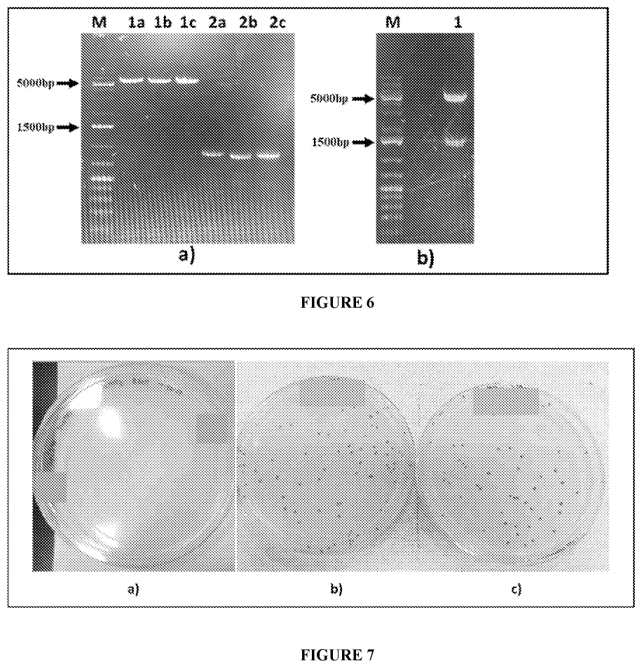

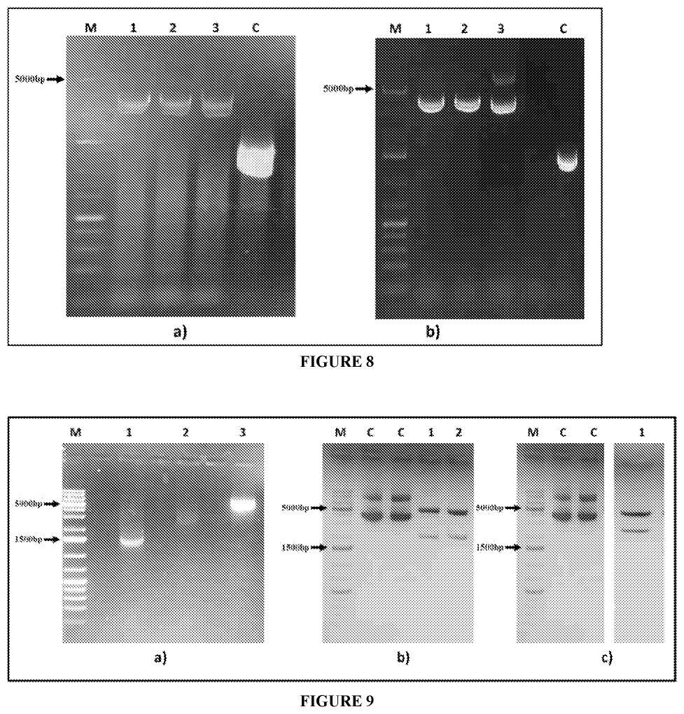

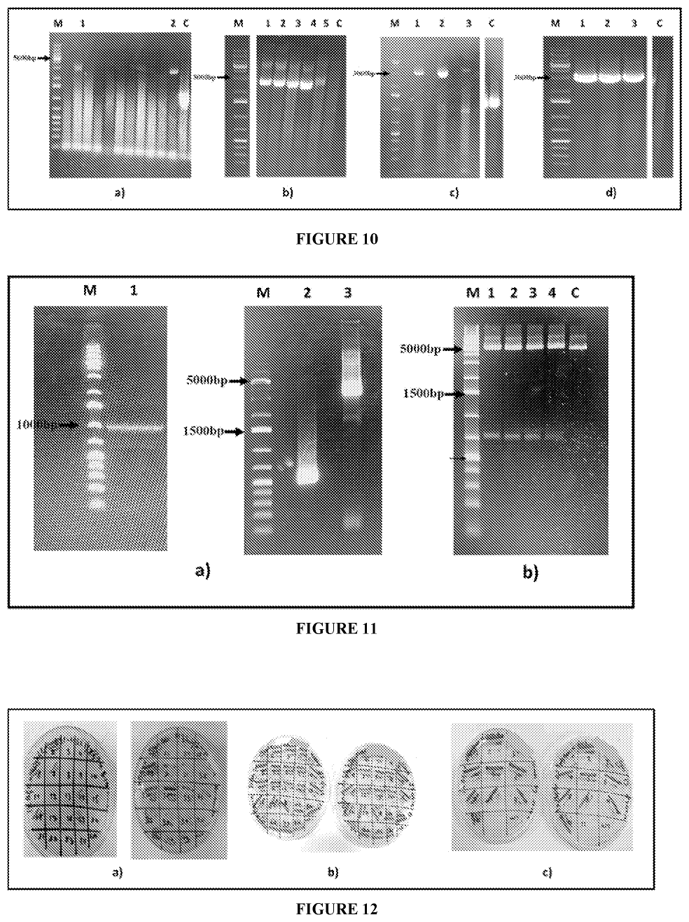

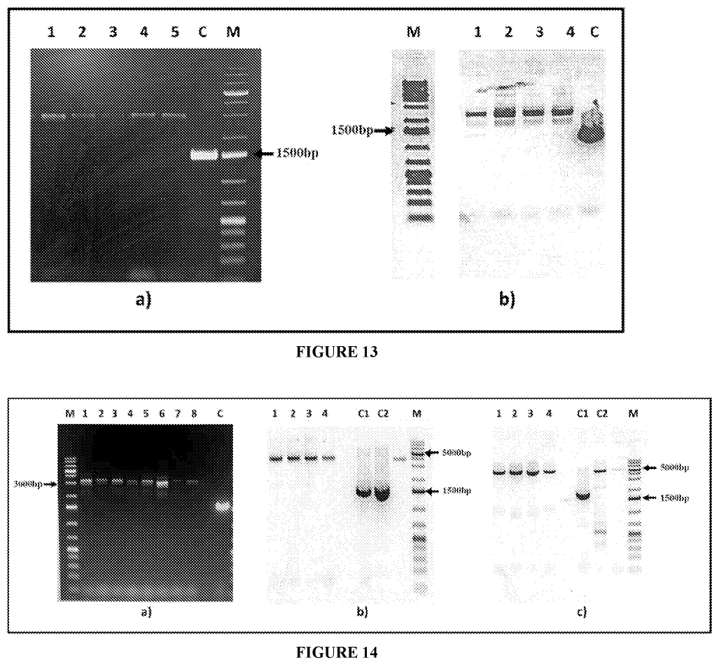

In some embodiments of the present disclosure, the recombinant algae comprises at least one motif of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1.

In some embodiments of the present disclosure, the recombinant algae comprises at least two motifs of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1.

In some embodiments of the present disclosure, the recombinant algae comprises at least one motif of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, at least two synthetic nucleic acid sequences that encodes tRNA specifying glycine amino acid and at least one synthetic nucleic acid sequence that encodes serine hydroxymethyl transferase (SHMT).

In some embodiments of the present disclosure, the recombinant algae comprises at least two motifs of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, at least two synthetic nucleic acid sequences that encodes tRNA specifying glycine amino acid and at least one synthetic nucleic acid sequence that encodes serine hydroxymethyl transferase (SHMT).

In some embodiments of the present disclosure, the recombinant algae comprises at least two motifs of synthetic nucleotide sequence encoding spider silk protein, wherein each motif of the synthetic nucleotide sequence encoding the spider silk protein comprises 10 copies of the monomeric units of MaSp1 illustrated in FIG. 1, at least two synthetic nucleic acid sequences that encodes tRNA specifying glycine amino acid and one synthetic nucleic acid sequence that encodes serine hydroxymethyl transferase (SHMT).

In some embodiments of the present disclosure, the recombinant algae comprises at least two motifs of synthetic nucleotide sequence encoding spider silk protein, wherein each motif of the synthetic nucleotide sequence encoding the spider silk protein comprises 18 copies of the monomeric unit of MaSp1 illustrated in FIG. 1, at least two synthetic nucleic acid sequences that encodes tRNA specifying glycine amino acid and at least one synthetic nucleic acid sequence that encodes serine hydroxymethyl transferase (SHMT).

In another exemplary embodiment of the present disclosure, the recombinant algae comprises at least two motifs of synthetic nucleotide sequence encoding spider silk protein, wherein each motif of the synthetic nucleotide sequence encoding the spider silk protein comprises 36 copies of the monomeric unit of MaSp1 illustrated in FIG. 1, at least two synthetic nucleic acid sequences that encodes tRNA specifying glycine amino acid and at least one synthetic nucleic acid sequence that encodes serine hydroxymethyl transferase (SHMT).

In some embodiments of the present disclosure, in the recombinant algae, at least one motif of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, at least two synthetic nucleic acid sequences that encodes tRNA specifying glycine amino acid and at least one nucleic acid sequence that encodes serine hydroxymethyl transferase (SHMT) encoding nucleotide sequence are integrated into the genome of the algae, wherein the integration of the said synthetic nucleic acid sequences are at specific locations in the genome of the algae for effective production of spider silk protein.

In some embodiments of the present disclosure, in the recombinant algae, at least two motifs of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, at least two synthetic nucleic acid sequences that encodes tRNA specifying glycine amino acid and at least one nucleic acid sequence that encodes serine hydroxymethyl transferase (SHMT) encoding nucleotide sequence are integrated into the genome of the algae, wherein the integration of the said synthetic nucleic acid sequences are at specific locations in the genome of the algae for effective production of spider silk protein.

In some embodiments of the present disclosure, the said motif of the synthetic nucleotide sequence encoding spider silk protein is integrated at the locus having sequence selected but not limited to sequence set forth as SEQ ID No. 22 and SE ID No. 23 in the genome of the algae. Further, the at least two synthetic nucleic acid sequences that encodes tRNA specifying glycine amino acid and at least one synthetic nucleic acid sequence that encodes serine hydroxymethyl transferase (SHMT) are integrated at the locus having sequence selected but not limited to sequence set forth as SEQ ID No. 24 in the genome of the algae. Thus, the synthetic nucleotide sequences of MaSp1, tRNA specifying glycine amino acid and SHMT are integrated into the genome of the algae to obtain recombinant algae of the present disclosure and that the said sequences are not provided as part of an extra chromosomal genetic material.

In some embodiments of the present disclosure, the said motif of the synthetic nucleotide sequence encoding spider silk protein, the nucleic acid sequence encoding tRNA specifying glycine amino acid or nucleic acid sequence encoding SHMT is integrated at the locus having sequence having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97%, at least 98% or at least 99%, similarity to the sequences selected from a group comprising sequences set forth as SEQ ID No. 22, SEQ ID No. 23 and SEQ ID No. 024.

In some embodiments of the present disclosure, the inclusion of nucleic acid sequence encoding tRNA specifying glycine amino acid and nucleic acid sequence encoding SHMT in the recombinant algae leads to about 5% to 15% higher glycine content when compared to the wild type algae. This higher content of the glycine content in the recombinant algae provides for stable production of spider silk protein in the recombinant algae.

The present disclosure further relates to method of producing the said recombinant algae.

In some embodiments of the present disclosure, the method of producing the recombinant algae comprises—
preparing an expression construct comprising a synthetic nucleotide sequence operably linked to a promoter;
transforming algal cells with the said expression construct to obtain recombinant algae.

In some embodiments of the present disclosure, the method of producing the recombinant algae comprises—
preparing an expression construct comprising motifs of the synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, operably linked to a promoter; and
transforming algal cell with the said expression construct to obtain recombinant algae.

In some embodiments of the present disclosure, the method of producing the recombinant algae comprises—
preparing an expression construct comprising synthetic nucleotide sequences that encode tRNA specifying glycine amino acid, synthetic nucleotide sequence that encodes serine hydroxymethyl transferase and at least one motif of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, operably linked to a promoter, respectively; and
transforming algal cell with the said expression construct to obtain recombinant algae.

In some embodiments of the present disclosure, the method of producing the recombinant algae comprises—
preparing an expression construct comprising synthetic nucleotide sequences that encode tRNA specifying glycine amino acid, synthetic nucleotide sequence that encodes serine hydroxymethyl transferase and at least two motifs of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, operably linked to a promoter, respectively; and
transforming algal cell with the said expression construct to obtain recombinant algae.

In some embodiments of the present disclosure, the method of producing the recombinant algae comprises—integrating expression construct comprising at least the one motif of synthetic nucleotide sequence encoding spider silk protein and expression construct of synthetic nucleotide sequences that encode tRNA specifying glycine amino acid and synthetic nucleic acid sequence that encodes serine hydroxymethyl transferase, to the genome of the algae.

In some embodiments of the present disclosure, the method of producing the recombinant algae comprises—integrating the expression construct comprising at least one motif of the synthetic nucleotide sequence encoding spider silk protein at the locus having sequence selected but not limited to sequence set forth as SEQ ID No. 22 and SEQ ID No. 23 in the genome of the algae and integrating the expression construct of synthetic nucleotide sequences that encode tRNA specifying glycine amino acid and synthetic nucleotide sequence that encodes serine hydroxymethyl transferase (SHMT) at the locus having sequence selected but not limited to sequence set forth as SEQ ID No. 24 in the genome of the algae.

In some embodiments of the present disclosure, the method of producing the recombinant algae comprises-creating a stressed environment for the algal cells to enable uptake of the expression construct of synthetic nucleotide sequence encoding spider silk protein and the expression construct of synthetic nucleotide sequences that encode tRNA specifying glycine amino acid and synthetic nucleotide sequence that encodes serine hydroxymethyl transferase (SHMT).

In some embodiments of the present disclosure, the expression construct comprising at least one motif of the synthetic nucleotide sequence encoding spider silk protein is an expression vector comprising motifs of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, promoters having sequences including but not limited to sequence set forth as SEQ ID No. 16, SEQ ID No. 17, SEQ ID No. 18, SEQ ID No. 19, SEQ ID No. 20, SEQ ID No. 21, and SEQ ID 25, and selection marker, wherein the vector backbone of the expression construct is compatible in the algal cells and is selected from the sequences including but not limited to sequence set forth as SEQ ID No. 3, SEQ ID No. 4 and SEQ ID No. 5.

In some embodiments of the present disclosure, the monomeric unit of MaSp1 illustrated in FIG. 1 is subjected to multimerization to obtain a motif of synthetic nucleotide sequence encoding the spider silk protein, wherein the motif comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1 (Foo, et al., 2006)

In some embodiments of the present disclosure, the expression construct of synthetic nucleotide sequences that encode tRNA specifying glycine amino acid and synthetic nucleotide sequence that encodes serine hydroxymethyl transferase is an expression vector comprising at least two synthetic nucleotide sequences that encode tRNA specifying glycine amino acid and at least one synthetic nucleotide sequence that encodes serine hydroxymethyl transferase, promoters having sequence including but not limited to sequence set forth as SEQ ID No. 16, SEQ ID No. 17 and SEQ ID No. 18 and selection marker, wherein the vector backbone of the expression construct is compatible in the algal cells, and is selected from sequences including but not limited to sequence set forth as SEQ ID No. 3, SEQ ID No. 4, SEQ ID No. 5.

In some embodiments of the present disclosure, the method of growing the recombinant algae comprises culturing the transformed algal cells at a temperature of about 25° C. to 40° C. under about 0% to 5% (v/v) carbon dioxide fortified in air, preferably at a temperature of about 35° C. under 2% (v/v) carbon dioxide for selection of the recombinant algae.

The present disclosure further relates to a method of production of recombinant silk protein by the recombinant algae of the present disclosure, wherein the production is a result of expression of the gene encoding the spider silk protein in the recombinant algae under the influence of promoter having sequences including but not limited to sequence set forth as SEQ ID No. 16, SEQ ID No. 17, SEQ ID No. 18, SEQ ID No. 19, SEQ ID No. 20, SEQ ID No. 21 and SEQ ID No. 25.

In some embodiments of the present disclosure, the production of recombinant silk protein by the recombinant algae of the present disclosure is a result of expression of the gene encoding the spider silk protein in recombinant algae, which comprises-expression of at least one motif of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, synthetic nucleic acid sequences that encode tRNA specifying glycine amino acid and synthetic nucleic acid sequence that encodes serine hydroxymethyl transferase (SHMT).

In some embodiments of the present disclosure, the production of recombinant silk protein by the recombinant algae of the present disclosure is a result of expression of the gene encoding the spider silk protein in recombinant algae, which comprises-expression of at least two motifs of synthetic nucleotide sequence encoding spider silk protein, wherein each motif of the synthetic nucleotide sequence encoding the spider silk protein comprises 1 copy, 2 copies, 3 copies, 4 copies, 5 copies, 6 copies, 7 copies, 8 copies, 9 copies, 10 copies, 11 copies, 12 copies, 13 copies, 14 copies, 15 copies, 16 copies, 17 copies, 18 copies, 19 copies, 20 copies, 21 copies, 22 copies, 23 copies, 24 copies, 25 copies, 26 copies, 27 copies, 28 copies, 29 copies, 30 copies, 31 copies, 32 copies, 33 copies, 34 copies, 35 copies, 36 copies, 37 copies, 38 copies, 39 copies or 40 copies of the monomeric unit of MaSp1 illustrated in FIG. 1, synthetic nucleic acid sequences that encode tRNA specifying glycine amino acid and synthetic nucleic acid sequence that encodes serine hydroxymethyl transferase (SHMT).

In some embodiments of the present disclosure, the method of production of the recombinant silk protein comprises—
preparing an expression construct comprising a synthetic nucleotide sequences operably linked to a promoter;
transforming algal cells with said expression construct;
selecting transformed algal cells;
culturing said selected algal cell in a suitable culture medium under conditions permitting the production of said recombinant protein;
recovering the recombinant silk protein from said culture medium; and
optionally purifying said recovered protein.

In some embodiments of the present disclosure, culturing comprises growing the selected algal cells (recombinant algae) in a medium including but not limited to BG11 medium (Gibco® 1X BG-11 Media with 20 mM NaHCO₃) and UPA medium, (Urea-1 mM, phosphoric acid-0.21 mM, and 2× trace element mix (FeCl₃·6H₂O −23.3 μM, Na₂EDTA·2H₂O −23.4 μM, CuSO₄·5H₂O −78.6 nM, Na₂MoO₄·2H₂O −52.0 nM, ZnSO₄·7H₂O −0.153 μM, CoCl₂·6H₂O −84.0 nM, MnCl₂·4H₂O −1.82 μM with 4% salinity).

In some embodiments of the present disclosure, recovering the recombinant silk protein comprises harvesting the cultured algal cells by technique including but not limited to centrifugation, followed by processing the extract/pellet obtained after harvesting.

In some embodiments of the present disclosure, the production of recombinant silk protein by the recombinant algae comprises-integration of the expression construct comprising at least one motif of the synthetic nucleotide sequence encoding spider silk protein and the expression construct of synthetic nucleotide sequences that encode tRNA specifying glycine amino acid and synthetic nucleotide sequence that encodes serine hydroxymethyl transferase (SHMT) in the genome of algae and culturing the recombinant algae in a suitable culture medium enabling production of recombinant silk protein.

In some embodiments of the present disclosure, the production of recombinant silk protein by the recombinant algae comprises-integration of the expression construct comprising at least two motifs of the synthetic nucleotide sequence encoding spider silk protein and the expression construct of synthetic nucleotide sequences that encode tRNA specifying glycine amino acid and synthetic nucleotide sequence that encodes serine hydroxymethyl transferase (SHMT) in the genome of algae and culturing the recombinant algae in a suitable culture medium enabling production of recombinant silk protein.

In some embodiments of the present disclosure, the production of recombinant silk protein by the recombinant algae of the present disclosure is simple and more economical when compared to the expression of the spider silk protein through fermentation in the bacterial system. The recombinant algae of the present disclosure can efficiently, and cost effectively convert solar energy into biomass. They require minimum nutrients to grow unlike the bacterial systems and also aid in carbon dioxide sequestration which makes the recombinant algae of the present disclosure a highly sustainable source for production of recombinant proteins, such as recombinant spider silk protein, described in the present disclosure.

The present disclosure further relates to use of the above described recombinant algae for production of recombinant silk protein.

In some embodiments of the present disclosure, in the said use, the production of silk protein is through the improved expression of glycyl-tRNA and the GlyA-serine hydroxymethyltransferase gene and the expression of at least one motif of the synthetic nucleotide sequence encoding the spider silk protein in the genome of the recombinant algae of the present disclosure.

The present disclosure further relates to use of the recombinant spider silk protein produced by the recombinant algae.

The recombinant spider silk protein produced by the recombinant algae may be spun into a fiber, or otherwise incorporated or formed into a fiber by any means known in the art, whether the means are presently known or developed in the future. The recombinant silk protein may also be used to make powders or thin films for use in coatings of medical devices or for other uses.

Further, the recombinant spider silk protein produced by the recombinant algae may be useful in artificial tendons and ligaments, bullet proof vests, airbags, athletic gear, parachutes, aircraft landing cables, biomedical sutures, drug delivery systems, cables, and other products where the properties of recombinant spider silk protein fibers may be desired.

Further, the recombinant spider silk protein produced from the recombinant algae may be used as an ingredient in personal care products such as skin lotion, shampoos, hair care products and medical applications such as wound dressing, sutures and as substrate for skin/cell culture.

Sequences referred to herein under SEQ ID No. 1 to SEQ ID No. 27 are captured in the sequence listing.

While the present disclosure is susceptible to various modifications and alternative forms, specific aspects thereof have been shown by way of examples and drawings and are described in detail below. However, it should be understood that it is not intended to limit the invention to the particular forms disclosed, but on the contrary, the intention is to cover all modifications, equivalents, and alternatives falling within the spirit and the scope of the invention as defined by the appended claims.

### EXAMPLES

The present disclosure is further described with reference to the following examples, which are only illustrative in nature and should not be construed to limit the scope of the present disclosure in any manner.

### Materials

Cyanobacterium aponinum was obtained from Gagva, Jamnagar, Gujarat.
E. Coli was procured from New England Biolabs

### Example 1: Development of Recombinant Cyanobacterial Host Having Improved Glycine Production

An extensive bioinformatic analysis was carried out to understand that C.aponinum genome has two copies of a glycyl-tRNA sequences. The tRNAs, called as glycyl-tRNA1 having sequence set forth as SEQ ID No. 9 & glycyl-tRNA2 having sequence set forth as SEQ ID No. 10, are about 71 bp and about 72 bp long, respectively. A GlyASHMT gene having sequence set forth as SEQ ID No. 11 was also identified from the C.aponinum genome. This enzyme was involved in the production of glycine from L-serine.

Said sequences SEQ ID No. 9, SEQ ID No. 10 and SEQ ID No. 11 were retrieved from the whole genome data bank of C.aponinum.

### Design of Construct for Overproduction of Glycine:

An in-silico integrative construct was designed with two tRNAs having sequences set forth as SEQ ID No. 9 and SEQ ID No. 10 and one GlyASHMT sequence having sequence set forth as SEQ ID No. 11 under influence of promoter having sequences set forth as SEQ ID No. 16, SEQ ID No. 17, SEQ ID No. 18 for integration at the locus of the genome of cyanobacteria having sequence set forth as SEQ ID No. 24.

### PCR Synthesis of Various Fragments:

Different templates were used for PCR synthesis of various fragments using commercially available DNA polymerase. The tRNAs, spacers and GlyASHMT genes were in vitro PCR synthesized from the genomic DNA template of C.aponinum. Promoters were synthesized as long primers. All fragments were cloned together using standard non-restriction enzyme based method. Upon preparation of the construct, correct assembly was further ensured by amplification of various junctions and restriction mapping.

### Induction of Natural Competency in Cyanobacteria and Transformation:

Natural DNA uptake method was used to deliver the glycine over-producing construct in C.aponinum. Briefly, a stressed environment with low light and low aeration was created for the cyanobacteria cells so that they could take up the foreign DNA (glycine over expression construct). About 2 μg plasmid was methylated using CpG methylase and transformed in cyanobacteria. BG11 media supplemented with about 100 μgmL⁻¹ Kanamycin antibiotic was used to screen the positive clones primarily, and for sub-culturing to get successful segregation of clones. To ensure the genome integration, genotyping method was used.

Post transformation and passaging, positive clones were evaluated for glycine production using the glycine assay kit. A comparative data analysis was done with indigenous strain of C.aponinum and the putative glycine-overproducing clones. One of the clones was found to produce 8% higher glycine over wild-type C.aponinum.

### Example 2: Cloning of MaSp1 Multimers in Cyanobacterial Vector

The structural gene encoding major ampullate spidroin protein (MaSp1) was custom synthesized. The synthesised MaSp1 monomer sequence is provided in FIG. 1. For initial cloning and gene assembly procedures, E. coli was used as the host chassis. The E. coli cells were purchased from NEB. The constructs generated thereof were excised from the E. coli and cloned in the cyanobacterial vector for expression of spidroin in C.aponinum strain (Location Gagva, Jamnagar, Gujarat)

For construction of concatemers of MaSp1, individual monomers were multimerized using restriction enzyme based cloning. (Prince et al., 1995) The colonies obtained were confirmed by restriction digestion after plasmid extraction.

### Cloning and Expression of Decamer (MaSp1-10 Copies) in C.Aponinum:

The decamer fragment (10 copies of MaSp1 monomer) was amplified from the sequence set forth as SEQ ID No. 6 (E. coli expression system) and using non-restriction enzyme based cloning with vector set backbone set forth as SEQ ID No. 4 for obtaining the final sequence set forth as SEQ ID No. 12. The colonies obtained were screened for gene insertion and segregation. Two cyanobacterial host chassis were used: Wild type strain and Glycine overproducing strain (GlyASHMT).

### Cloning and Expression of Octadecamer (MaSp1-18 Copies) in C.Aponinum:

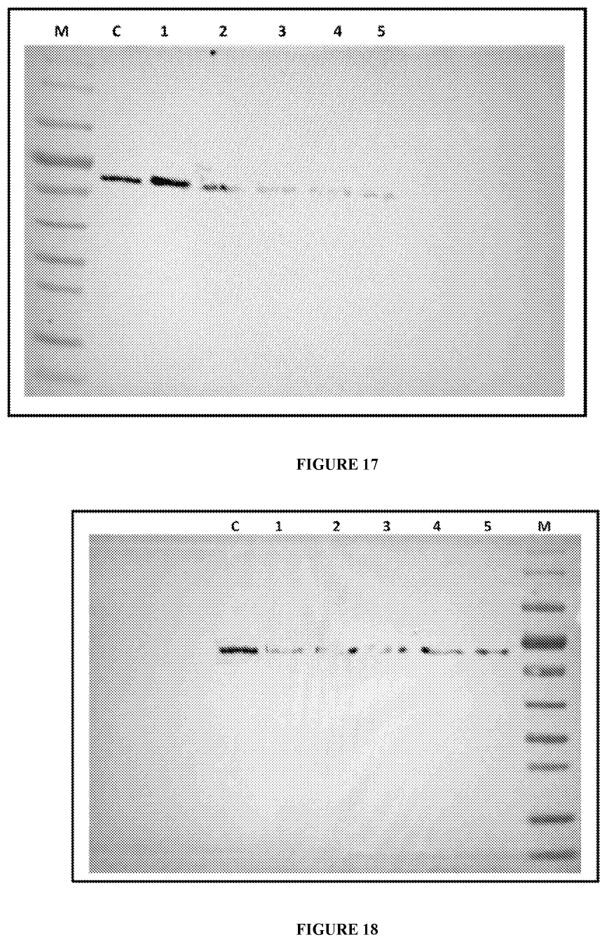

The octadecamer fragment (18 copies of MaSp1 monomer) was amplified from sequence set forth as SEQ ID No. 7 (E. coli expression system) and using non-restriction enzyme based cloning with vector backbone set forth as SEQ ID No. 4, the final sequence set forth as SEQ ID No. 13 was constructed. The colonies obtained were screened for gene insertion and segregation. Two cyanobacterial host chassis were used: Wild type strain and Glycine overproducing strain (GlyASHMT). FIG. 17 illustrates western blot analysis of the strain expressing one copy of the octodecamer MaSP1 in GlyASHMT C. aponinum, wherein Lane M=Protein Marker, Lanes 1-5=western blots of putative transformants, Lane C=western blot of decamer MaSP1 in GlyASHMT C. aponinum.

### Cloning and Expression of Trigintahexamer (MaSp1-36 Copies) in C.Aponinum:

The trigintahexamer fragment (36 copies of MaSp1 monomer) was amplified from sequence set forth as SEQ ID No. 8 (E. coli expression system) and using non-restriction enzyme based cloning with vector backbone set forth as SEQ ID No. 4, the final sequence set forth as SEQ ID No. 14 was constructed. The colonies obtained were screened for gene insertion and segregation. Two cyanobacterial host chassis were used: Wild type strain and Glycine overproducing strain (GlyASHMT). FIG. 18 illustrates Western blot analysis of a strain expressing one copy of the trigintahexamer MaSp1 in GlyASHMT C.aponinum, where Lane M=Protein Marker, Lanes 1-5=western blots of putative transformants, Lane C=western blot of decamer MaSp1 in GlyASHMT C.aponimum.

The procedure described in Example 2 can be employed for expression of other similar spider silk proteins selected from a group comprising Major Ampullate Spidroin-2 (MaSp2), Minor Ampullate Spidroin-1 (MiSp1), Minor Ampullate Spidroin-2 (MiSp2) and Flagelliform (Flag).

### Example 3: Cloning and Expression of Two Motif Having-10 Copies of MaSp1 Monomeric Unit

In this experiment, the copy number of spidroin cassette (construct) in glycine overproducing strain was increased and the effect of integration site on transgene expression was demonstrated. The decamer fragment of MaSp1 (10 copies of MaSp1 monomeric unit) was amplified from sequence set forth as SEQ ID No. 6 and using non-restriction enzyme based cloning with vector backbone set forth as SEQ ID No. 5, the final sequence set forth as SEQ ID No. 15 (two motifs having 10 copies of MaSp1 monomeric unit) was constructed. The colonies obtained were screened for gene insertion and segregation.

The transformants obtained were also cross-checked for the presence of earlier two transgenes. As shown in FIG. 14, these two loci were found to be intact in all the transformants obtained. All the positive transformants were propagated in liquid medium for further growth and expression studies. The obtained transformant was submitted with the International Depository Authority on Jan. 5, 2021. The accession number is CCAP 1483/10 Cyanobacteria HSS 171.

### Example 4: Cloning and Expression of Decamer (MaSp1-10 Copies) in C.Aponinum

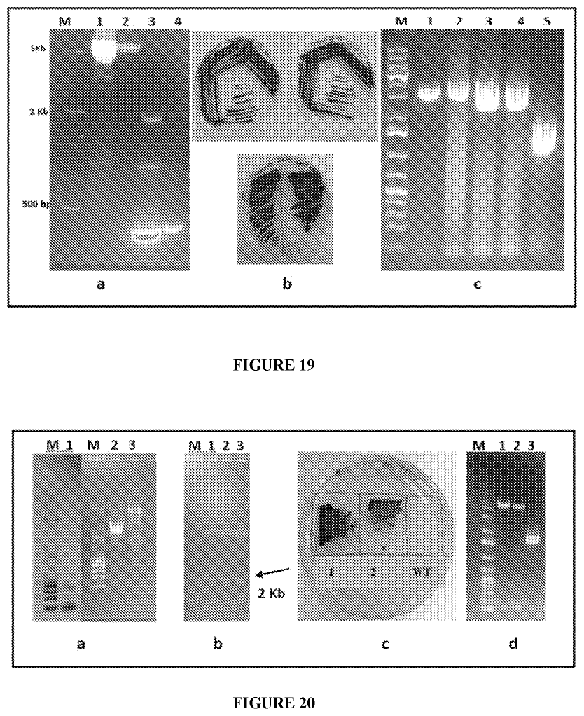

The decamer fragment (10 copies of MaSp1 monomer) was amplified from the sequence set forth as SEQ ID No. 6 (E. coli expression system) and cloned with the SEQ ID No. 21 and/or SEQ ID No. 25 using non-restriction enzyme based cloning with vector set backbone SEQ ID No. 4 for obtaining the final sequence set forth as SEQ ID No. 26 and/or SEQ ID No. 27. FIG. 19 provides the amplification of all the three fragments. The colonies obtained were screened for gene insertion and segregation. Two cyanobacterial host chassis were used: Wild type strain and Glycine overproducing strain (GlyASHMT). FIG. 20 provides the confirmation by colony PCR of the cyanobacterial transformants with the decamer MaSp1 construct with Seq ID No. 21 and/or Seq ID No. 25 in WT C.aponinum and GlyASHMT C.aponinum.

### Example 5: Expression Analysis of Spidroin Protein in Green Cyanobacterial Chassis

Respective C.aponinum clones obtained as per above Examples 2 and 3 were analyzed for gene expression and confirmation of spidroin protein expression. C.aponinum clones were analyzed by SDS PAGE coomassie staining and western blot with modification in induction conditions and sample preparation. The clones were allowed to grow in BG11 medium at a starter OD₇₅₀ of 0.6. At 2.0 OD₇₅₀, these cultures were shifted to UPA medium (1 mM Urea, 0.2 mM phosphate) supplemented with about 10 mM Na₂HCO₃. After about 2 days of growth at a temperature of about 35° C., urea was exhausted in the medium and additional nitrate was added at a concentration of 17.6 mM. The cells were grown for 3 days. Thereafter, the cells were harvested by centrifugation (15000 rpm, 20 min). Pellets were collected and processed for protein extraction. Pellets were washed initially with PBS and then further re-suspended in about 2 mL lysis buffer (100 mM sodium chloride (NaCl), 25 mM Tris HCl, pH 8.0) with protease inhibitor cocktail (ROCHE Complete™) and 0.1 mM PMSF. The cells were broken with glass bead betting for about 2 min in 3 cycles with intermediate cooling on ice to avoid excess temperature rising. The final cell lysate with final concentration of about 5 μg proteins was analyzed by SDS PAGE coomassie staining and western blot as mentioned previously. An antigenic polypeptide specific for spidroin was predicted using various Bioinformatics software. The peptide with sequence “SGRGGLGGQGAGAAC” was synthesized and used as antibody detection of spidroin expression.

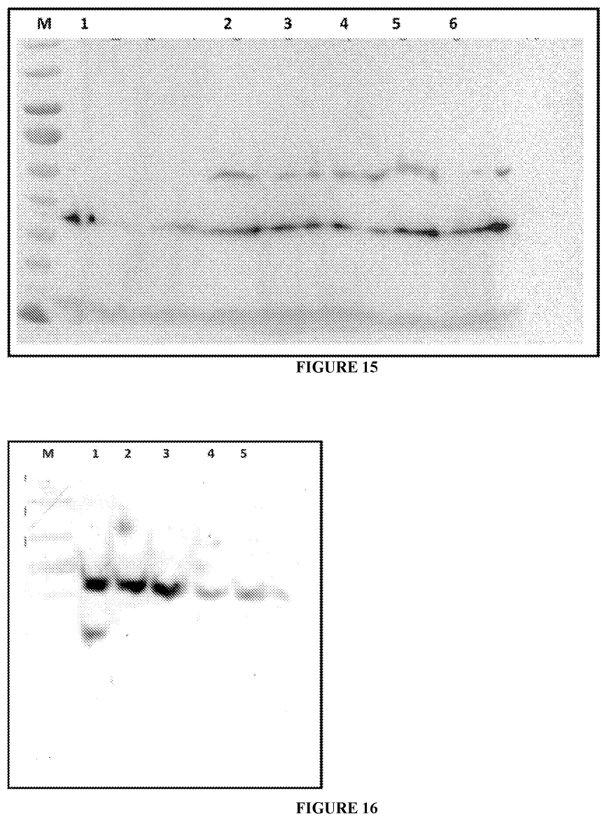

The expression of MaSp1-10× in WT C.aponinum and GlyASHMT C.aponinum strains was confirmed by western blotting (illustrated in FIG. 15). The increase in copy number of MaSp1-10× was also validated by the same protocol (illustrated in FIG. 16).

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

All references, articles, publications, general disclosures etc. cited herein are incorporated by reference in their entireties for all purposes. However, mention of any reference, article, publication etc. cited herein is not, and should not be taken as, an acknowledgment or any form of suggestion that they constitute valid prior art or form part of the common general knowledge in any country in the world.

### REFERENCES

1. Foo, C., Bini, E., Huang, J. et al. Solution behavior of synthetic silk peptides and modified recombinant silk proteins. Appl. Phys. A 82, 193-203 (2006).
2. Prince J T, McGrath K P, DiGirolamo C M, Kaplan D L. Construction, cloning, and expression of synthetic genes encoding spider dragline silk. Biochemistry. 1995 Aug. 29:34(34): 10879-85.

We claim:

1. Recombinant algae comprising:
at least one motif of synthetic nucleotide sequence encoding spider silk protein;
at least two synthetic nucleotide sequences encoding tRNA selected from a group having sequence set forth as SEQ ID No. 9, SEQ ID No. 10 and combination thereof; and
at least one synthetic nucleotide sequence encoding serine hydroxymethyl transferase (SHMT) having sequence set forth as SEQ ID No. 11.

2. The recombinant algae as claimed in claim 1, wherein the motif comprises multiple monomeric units of gene encoding spider silk protein.

3. The recombinant algae as claimed in claim 2, wherein the motif comprises 10 copies of the monomeric unit of the gene encoding the spider silk protein, wherein the motif is having sequence set forth as SEQ ID No. 12.

4. The recombinant algae as claimed in claim 2, wherein the motif comprises 18 copies of the monomeric unit of the gene encoding the spider silk protein, wherein the motif is having sequence set forth as SEQ ID No. 13.

5. The recombinant algae as claimed in claim 2, wherein the motif comprises 36 copies of the monomeric unit of the gene encoding the spider silk protein, wherein the motif is having sequence set forth as SEQ ID No. 14.

6. The recombinant algae as claimed in claim 1, wherein the recombinant algae comprises two motifs of the synthetic nucleotide sequence encoding the spider silk protein having sequence set forth as SEQ ID No. 15, wherein each motif comprises 10 copies of the monomeric unit of gene encoding the spider silk protein.

7. The recombinant algae as claimed in claim 1, wherein the at least one motif of synthetic nucleotide sequence encoding the spider silk protein is integrated in genome of the algae at locus having sequence set forth as SEQ ID No. 22, SEQ ID No. 23 and combination thereof

8. The recombinant algae as claimed in claim 1, wherein the at least two synthetic nucleotide sequences encoding tRNA and the at least one synthetic nucleotide sequence encoding serine hydroxymethyl transferase is integrated in genome of the algae at locus having sequence set forth as SEQ ID No. 24.

9. The recombinant algae as claimed in claim 1, wherein the tRNA is tRNA specifying glycine amino acid.

10. The recombinant algae as claimed in claim 1, wherein the algae is selected from a group comprising Chlamydomonas, Nanochloropsis, Chlorella and Cyanobacteria aponinum.

11. An expression construct comprising at least one motif of synthetic nucleotide sequence encoding spider silk protein, operably linked to promoter.

12. The expression construct as claimed in claim 11, wherein the motif comprises multiple monomeric units of gene encoding the spider silk protein.

13. The expression construct as claimed in claim 12, wherein the motif comprises 10 copies of the monomeric unit of the gene encoding the spider silk protein, having sequence set forth as SEQ ID No. 12: wherein the motif comprises 18 copies of the monomeric unit of the gene encoding the spider silk protein, having sequence set forth as SEQ ID No. 13; and wherein the motif comprises 36 copies of the monomeric unit of the gene encoding the spider silk protein, having sequence set forth as SEQ ID No. 14

14. The expression construct as claimed in claim 11, wherein the promoter is selected from a group having sequence set forth as SEQ ID No. 16, SEQ ID No. 17, SEQ ID NO. 18, SEQ ID No. 19, SEQ ID No. 20, SEQ ID No. 21, SEQ ID No. 25 and combination thereof.

15. An expression construction comprising at least two synthetic nucleotide sequences encoding tRNA selected from a group having sequence set forth as SEQ ID No. 9, SEQ ID No. 10 and combination thereof; and at least one synthetic nucleotide sequence encoding serine hydroxymethyl transferase (SHMT) having sequence set forth as SEQ ID No. 11, operably linked to promoter.

16. The expression construct as claimed in claim 15, wherein the promoter is selected from a group having sequence set forth as SEQ ID No. 16, SEQ ID No. 17, SEQ ID No. 18, SEQ ID No. 19, SEQ ID No. 20, SEQ ID No. 21, SEQ ID No. 25 and combination thereof.

17. A method of producing the recombinant algae as claimed in claim 1, said method comprises:
integrating the expression construct as claimed in claim 11 at locus having sequence selected from a group comprising sequence set forth as SEQ ID No. 22, SEQ ID No. 23 and combination thereof in genome of algae; and
integrating the expression construct as claimed in claim 15 at locus having sequence set forth as SEQ ID No. 24 in genome of algae to obtain the recombinant algae.

18. A method of producing spider silk protein, wherein said method comprises culturing the recombinant algae as claimed in claim 1 in a culture medium under condition suitable for producing the spider silk protein.

19. The method as claimed in claim 18, wherein the culture medium is selected from a group comprising BG11 medium, UPA medium and combination thereof.

20. The method as claimed in claim 18, wherein the condition comprises temperature ranging from about 25° C. to 40° C. and air fortified with carbon dioxide ranging from about 0.01% to 5%.


SEQUENCE LISTING (27 sequences)

SEQ ID NO: 1 - DNA, 96 bp, Artificial Sequence
  MaSp1 monomer unit
agcgggcgtggagggctgggggggcaaggtgcaggagcagccgcagccgctggaggtgccggacaggggggttatgggggattaggtagccagggt

SEQ ID NO: 2 - DNA, 5422 bp, Artificial Sequence
  Vector Sequence
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgatatcagccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 3 - DNA, 6082 bp, Artificial Sequence
  Vector Sequence
tctccgtttcttatctgtttgaattcacatacgcggccgcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgcccacggaaattattgttgatagctctttaggtaatcaaaacaatcctgatgtggatctcgctcctgatggtagtatcctcattagttataccgatgaaagttctaacagtgtgaaatatcgtcagtttaacagtaacggtgaacctctaggagatagtgccacttttgacgttgagggcaatcaagaaactaactctgccatagcagtaggaactaataataaagcagtagttgtcgctgatgcaggaaataacaatagttttaacccttatggtcaaattctaactcctgatgctagtaatactctcaatatccctttaaatcgctttcaaaataccagtcaacccggcacatatttatttgcaggggagcaagaaagtcaaaatattcgtcagaatttccccaactttgtggaagaaggatttgctttttctgtggcggaaaaccctcaagataacttaattcgttttaaccgttttcaaaacagcgatcgccccggtacatatttatttgcaggggagcaagaaagtcaaaatattcgtcagaatttccccaactttatcgaagaaggagtcgctttttatgcctttggtgcaggttcaaatcaagccactcctttttatcgtttccaaaatactgatgtacccggtacttacctttttgttggtgatagtgaaagacaaagtattcttcagaatttccccaattttgtagaagagggcattgcttttgaagcggctacttagttagcttaatcggcgagaagccccacgcttagttttcgacaaaaaaattaaacaagcgtgggacggggcttttaaagagcggaaatagttccgctcactgcccctcatgaatcgccagtgacgtgttataatgactaaaaagtggggctggacgtgtacctcactaaaaaacgccgtagtgggaaagaaggagtaccacgatggaagttcaagaaggataatttccgcttgaagcctacactctgcgtaagcaagtgtatggagcgtcactattcgccatcaacgagaatcaattgatttaatcatggttgtaggggaaaaactaccccagacaacatcaaacaaatgctcaacaaatgttattatcttttacataaattaagaagataaaaactggagaataaaaaaatggactggagagtgttaatcgtattattacaggacgcccgccataaactgccaggcatcaaattaagcagaaggccatcctgacggatggcctttttgcgtttctacaaactccggatccggccggatccggccaagcttgctagctcgatgactagtgtcactagcgatgttcgacatcgtggatgtcgttctcctcggggtgaaatgaggttatttgttttctgtagttctaacttacccacctcgatcgcaaatcatatcacctgccacaaaattttttttcaatttttccgggaaatcgtgtatcgcccacccagtggggaataccgactaaggcggttggggtgactgggtgtcagacatagggcttagggggttaaggccgccaccaattggtaaacctcgccaattttccctataactaactgttaaatgagatgaccctaaaaagatcttaacttccatcaagaaaataaaacaaggggtaaatctggaaaaaaaaaaaagacaatgatatactagaaaagttgtcaaaattcgcacatctaaggcatcgggtgtttctagccaaagaaattaaacaaccttagatggaggattaacccgaaaaaaggagaaaataatgagtcatattcaacgcgaaacgtcttgcagtaggccgaggctgaattctaatatggatgcagatttatatgggtataaatgggcacgcgataacgtaggtcaatctggagctactatttaccgattatatggtaaacccgatgcaccggaattatttttaaaacatggtaaaggatctgtggccaatgatgttactgatgaaatggttcgactcaattggttgactgaatttatgccattacctaccattaagcattttattcggactccagacgatgcatggttacttactactgcgattccgggtaaaacggcgtttcaagttttggaggaatatcctgattctggagagaatattgttgatgctttagcagtttttttgcgtcgcttgcattctattcccgtttgtaactgtcctttcaatagtgatcgcgtatttcgccttgcacaagcgcagtctagaatgaacaatgggttagttgatgcttccgattttgatgatgagcggaatggttggccagtagaacaagtttggaaagaaatgcacaaacttttaccctttagccccgattcggtcgttacccatggtgatttttccttggataatttgatcttcgatgaaggtaaacttataggttgtatcgatgttgggcgcgttggaatcgctgacagatatcaagatttagcgattctttggaattgtttaggtgaattttcacccagtctacaaaaacgcttatttcaaaaatatggtatagataatcctgatatgaataagttacaatttcatttaatgttagatgaatttttctagactagtctagctggcctggaagggtgggaaagtgttaacaactggttgacaatattccgcttttccaggtcttgtcgaagtacgcattatttaatattggtgctgctgcgatcgcacaattccaaaaagttctcaacaaacaaaattcttaacttcagaggctaaaacgcaagggggtaaaaccccttttttaactgagttcggaataaattttcatctatgaataatggcgaaaagggcaagaagcaaaggcaaaccctaacacctgaaacctgaaacctgaaacctacccttacccaatattcttaaaccgaactgaggttaaataagtcttcctcttttcaatccttaataaattttaacgatcgccgcttccatattgcaacaattccaaataacgatgatagtaatactgttgtcgaggagaaaatttttcagagaaaccaataaaacccttagcgaaaggatataaacccgtgtaaactccaagactaagataatgtttgagccaatcagctaaaatgggtaaacccacttgaggaagtaaaggtaaaactaacatcggattaactagcggtaaagtctttgttaagcccgaaaactgcaccacatcttgtaaaaaaggctttagcacatcgtcacctaatttatccattacccgaaaaacaccgctcattaaatcgttgatttgattaggttggggttgcttatcaataggcacactcatagttttttgaaataaccaagtaacagaaatatttggttgataaggctgaattaaagctaaatcctcacgggttaaataatctccttttaaggcttcatcaatggctaaacttaaacgaggtaaatgacgcatcatcgccccaaaaccaccaaaactgacaggagattgcataccactactatcccccaccgctataattctatcccaaggcatttttaaaggactttgacggtaagcggggaaaaaaccaaataaaaaccgttgaaaatcgagattatctaattctatttgttgatattcgggcagtaatcttaaatattccgtcattaagttttttaggctaattctttcagggttagcatccacataggtaaaaagataggttgtgcgtccatccttagctggaaaagcctcccagaaatattgacattgattaataatcggagtaaaagaataaattaaatctcctgtttcattctgtttaaatccttttccacaactaccgacaactaaacaaactccatcgggtttt

SEQ ID NO: 4 - DNA, 5946 bp, Artificial Sequence
  Vector Sequence
ggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcttgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcctgatttggttgacgtagtttttgaatcgcttttgcctgaatttgtctaactctttcccttgataaattcataattgaggcagtatcactaagggaataaattttgccattttcaaacccataacgcagtctcagaattttttgttctctttcattgagtaaagaaagcatagagcgcaaatccttttgcatagatactctcattaagttttcttcgggagttgcagattctgtttcaattaattccactaattcagtgttataatcttctcccactttcatttctaaggatatggagcggggtatttgatttaaaaattgacgcaaatcaagggaagtcatatccatttctttggctatttcatctactgtggcgattctgcccaatttttgggataattgtctctgtactttttttagttgatttaatctttcagtaatatggataggaagacgaatggttctagcttgagttgcgatcgctcttgtcattccctgacgaatccaccaataagcataagtgctgaaatgatagcctttggtatgatcaaatttttcaacggctttttctaaaccgagagtaccttcttgaattaagtctaataattctaatcctctattttgatattttttggcaatagagacaactaaacgcaaattagcattaatcattttctgttttgctttaatgccattagccaaaatagtttctaactctgatagtttgacttgtgctaaattagcccatgtttcttttccttctgtaattatcgattctacttttttcggagttaaattgagttgttgagcgatttttcctattttgactgcttcagagtgattcgctaccaaagaatcaaatattgtcatgatttccaaatattttgcaagaactggcagttccctatttttgtctctgattgccaataaattttgagaggccgtacttcgacaagacctggaaaagcggaatattgtcaaccagttgttaacactttcccacccttccaggccagctagactagtttattttcctacaacttttgtaatctcccccttcacgtagtgaacaaattcttctaactgatctgctctagaagctaacctatcttcctcttgtcctaaataagcttgacgtgcttccaaaattacgggttgatattgagcaggtaatcgttccatggcccaatcggctgccacgtctttaggagcaattttccctgtaactgcagaataccaaatacgagataaagtaagtacaacatttcgttcatcacctgcccagtcggggggactattccataaggttaaagtttcatttaaagcttcgaataagtcttgctcagggacagggtcaaataactcttcggcagcagggccaactaaggccacgctgtgttctctggctttggtaagaagaatcgctaaatcaatatcaatagtggcaggttcaaaaattcctgctaaaatatcattcctttgccattctccaaattgtaactcacgcttcgctggatatctccagggaataatatcgtcatgtactactatggtcacttcaactgcccgaagaatctcagattccccaggggatgcacttgtttctaacaaatcattaattaaggcacgacgagttgtttcatccaggcgaacggtgacagttacaagtaaatctatgtcactgtgaggttttaatcctccatctacagcagatccatataaatggacagctaataaggtgggttctaaatgacgttcgattactcctacaacttcagagagttgtgtgctaacctcggcaataacggcttcacgcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtacaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttgcttctcacactggctcaccttcgggtgggcctttctgcgtttatatactagagagagaatataaaaagccagattattaatccggcttttttattatttctaggacagcttaaccggtacatcaaaatacccaaatccttactctcccctcaatatacaacaaaaaaaactttttgattcactttagtcataaaaattagaatttatctaccgaaatattacataaatgtaatgtatatattttctgatttattccgtgtgagccatgattcataatttataattcataatttctaaatatgcccctacaatggatatagaatgtcattttaattataggtatcataatcgtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaagg

SEQ ID NO: 5 - DNA, 5301 bp, Artificial Sequence
  Vector Sequence
actcaggtagaagaaattaagaattcacatacgcggccgcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgccctctcttctacttcttccaatgcttctaatactctctcacgagtggcagatgcgatcgcaccattgatttcaattcgagcaatttgtttacgagtcgaaggtttaaaaggccaaatcattctttaagtatttctaaattttttttggttaacaattatatacatccaaacaactcattataacttacctactcattctcaacacaataataaatttataatttgcaattagtcaccaagaatttattgcaaacctgaaaaaagagtgagaattaaaaactcctaacactcattactgattacttaacctgagttttggataagctgaaagcagaccaactcgtaatcagaaaaccttatagcttcttctcagaaataacgataaaattgccctaacccgaactgacgttaaatatgttcaacccctacttcaccctctcaccctctccccttatctccctaacacctgcaacctgacacctaaccttatcggatattcttaaaccgaactgaggttacttattacttgttcctccctcgtcttgctatccccaacacctgacaccactttttcaacaattcctaatttgtcttcgccttttataactaagaatggcatccacaaccgctatggccacatccttctccttccttatgtccttcggcacaagccgttgagcaataataatgcccatctctggaaattgcttgactaatatcaattatacataaacaagagggacaagcacatttcatttgagttactgtagtggtagtcataatcaaaattctcctatctgtgaatatctgaatataaattcagtagttgtctaataattagattaacacaaaatatgatttattgttcatttgttaatatattttacaaacaaccataaaattagtagctatatttcagagagtaatgattcattacaagatctaggaataggagaaaaagaaaaatacaatagggttaagctgtcctagaaataataaaaaagccggattaataatctggctttttatattctctctctagtatataaacgcagaaaggcccacccgaaggtgagccagtgtgacgccaattttccctataactaactgttaaatgagatgaccctaaaaagatcttaacttccatcaagaaaataaaacaaggggtaaatctggaaaaaaaaaaaagacaatgatatactagaaaagttgtcaaaattcgcacatctaaggcatcgggtgtttctagccaaagaaattaaacaaccttagatggaggattaacccgaaaaaaggagaaaataatgctaaggagttctaatgatgtgacccaacaaggatcaaggcctaaaacaaaactggggggatcttctatgggtattattaggacttgtagacttggtcctgaccaagtgaaatcaatgcgtgcagccttagatttatttggacgcgagtttggggatgtggcaacctattctcagcatcaacccgatagtgactatctaggaaatttacttagaagcaaaacgtttattgctttggctgcgtttgatcaggaggcagtcgttggtgcgttagctgcatatgttttacctaaatttgagcagcctcgttctgaaatttatatctatgatttagcagtttctggggaacatagacgtcaaggaattgccacagccttaatcaatttattgaaacatgaagcaaatgcattaggggcgtatgtaatttatgtgcaggctgactacggtgatgaccctgccgttgcactttatacgaaactaggtatcagagaagaagtaatgcattttgatatagatccttctacagctacttagactagtctagctggcctggaagggtgggaaagtgttaacaactggttgacaatattccgcttttccaggtcttgtcgaagtacggcatcaatgggaaaaatataaacaagagtcgatcgaaatgttgcaatactggacaaatcaggcaaaagatcaagggctaaaagcagaatttaatcaaatttacgggcatccgggtactattatttgtcaacaggctcaggaatggggagcaaatctaattgttatgggtagaagaggacattctaccgtgagcgaattattattaggcagtgtcagtagttatgttattcaccgcagtcattgtgcagtacatttggtacagttttaagatattggaaagtttggagtagtgaatagaaatagggaggtttcaggtagcaggtagcaggtagcaggtaagaggtaacaggtttaagaatctcaaacattgattccaaatgtcaaaggttttacataaataattcaaatgtcgctcattaacgcccttgtacgcccatacttcaatagactcttgtacaagaataaagaagaaaatcaataaacatcatatcaaatctatttcttgtcaactctttaacccgacacctgacacctaaaaccaatcatgtattctatttttgcctaacatcagttattcactattcatctttgccctcccccctctcgaggggggataaagggggatttgccccttgccctttacctttgattgctaattgttaattgctaattgaaatattgatggtaaattattaaaagagtaacgataaaataaccaactcagtcaatttagtaacaaggattcacgggtggaaaaaggaacattagttgaatttaaagtaaatggcgatcgccgcttaggtgtcattgagaaaccagaagggaaaaaagactggatagccatagatgaaaacggcaattcccacaaagttagaccccaaaaagtagattttttagtagaaggagaatcctttaaatataccgatattccccgatttgtaaaagaagtaacccctcatttagatgcttctagcttggaaattgcttgggagttattgattgaagaagcaactcctgttactccggctgaattagcaactctcatttttgaggaggaaacaccggttttctgttatgccgcccatttacttttatctgatgacaagatttattttaagaaaaaaggggatatttatgaacctcgtcctcaa

SEQ ID NO: 6 - DNA, 6457 bp, Artificial Sequence
  E.coli expression system
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccactagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagccatggatccgatatcagccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 7 - DNA, 7273 bp, Artificial Sequence
  E.coli expression system
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccactagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagccatggatccgatatcagccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 8 - DNA, 9109 bp, Artificial Sequence
  E.coli expression system
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccactagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagtaccctggctacctaatcccccataacccccctgtccggcacctccagcggctgcggctgctcctgcaccttgcccccccagccctccacgcccgctgctagccatggatccgatatcagccatggccttgtcgtcgtcgtcggtacccagatctgggctgtccatgtgctggcgttcgaatttagcagcagcggtttctttcataccagaaccgcgtggcaccagaccagaagaatgatgatgatgatggtgcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 9 - DNA, 95 bp, Artificial Sequence
  tRNA
gcgggtgtagtttagtggtaaaaccttagccttccaagctaatgataggggttcgattcccctcacccgcttatcaatcttttatcctctcatct

SEQ ID NO: 10 - DNA, 96 bp, Artificial Sequence
  tRNA
gcgggtatagctcagtggtagagcgtcaccttgccaaggtgaatgtcgcgcgttcgaatcgcgttacccgctttaatggtttttacccctaatatt

SEQ ID NO: 11 - DNA, 1284 bp, Artificial Sequence
  serine hydroxymethyl transferase
gtgagtaaaactaacttagaatttttagccgacacagatccagctatagcggaaattattgctaatgagttacaaagacaaagaggacatttagaattaattgctagtgaaaattttacctctcctgcggtaatggcggcacagggttctgttttaaccaataagtatgcagagggtttaccgggtaaacgctactatggtggttgtaaattcgttgatcaagcggaagatttagccattgaaagagcaaaacagttatttggggcggcaatggcaaatgtacaaccccattcaggggcgcaggctaattttgctgtatttttaacccttcttaaccccggggataaaatcatggggatggatttatctcacggtggacatttaactcatggttctcctgttaatgtttctggtaaatggtttgaggtttgtcaatacggtgtaagtaaagaaactgaacgccttgactatgatcaaattcgtgagttagcgttaaaagaacgtcctaagttaatcatttgcggttactccgcttatcctcgcattattgattttgaaaaattccgtgcgatcgcagatgaagtaggagcgtatttattagcagatattgcccacattgcaggattagtagccactgggcatcatcctaaccccgtgcctcattgtgacgtagttactaccaccacccataaaactctcagaggacctcgtggaggcttaattttgacccgtgacgcagaattaggtaaaaaattaaataaatctgtttttcccggtactcaaggcggtcctttagaacacgtcattgcaggtaaagcagtggcttttggtgaagctttaaaacctgagtttaaaacctattgcggacaagttatcgccaattctaaagcattgggtaatcagttagtaaatagaggctttaagctagtttctggcggtagtgataatcacctcaatttagtggatttacgctctatcggcatgacgggtaaagtggcagatcaacttttaggagaaatcagtattactgcgaacaaaaatactgtaccttttgatcctgaatctccttttgtcaccagtggtattcgtttgggaactcctgctgaaaccactagaggtttaaaagaagaggatttcaccgaagtggctaatattatcgctgattgtttactcaatcctgatgatgcagatgtgaaagctaattgtattaagagagtagaggcattatgcgatcgctatcctctttatcctcatttacagattcctgtacctgctttagcttaa

SEQ ID NO: 12 - DNA, 7206 bp, Artificial Sequence
  10 copies of monomeric unit of MaSp1
ggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcttgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcctgatttggttgacgtagtttttgaatcgcttttgcctgaatttgtctaactctttcccttgataaattcataattgaggcagtatcactaagggaataaattttgccattttcaaacccataacgcagtctcagaattttttgttctctttcattgagtaaagaaagcatagagcgcaaatccttttgcatagatactctcattaagttttcttcgggagttgcagattctgtttcaattaattccactaattcagtgttataatcttctcccactttcatttctaaggatatggagcggggtatttgatttaaaaattgacgcaaatcaagggaagtcatatccatttctttggctatttcatctactgtggcgattctgcccaatttttgggataattgtctctgtactttttttagttgatttaatctttcagtaatatggataggaagacgaatggttctagcttgagttgcgatcgctcttgtcattccctgacgaatccaccaataagcataagtgctgaaatgatagcctttggtatgatcaaatttttcaacggctttttctaaaccgagagtaccttcttgaattaagtctaataattctaatcctctattttgatattttttggcaatagagacaactaaacgcaaattagcattaatcattttctgttttgctttaatgccattagccaaaatagtttctaactctgatagtttgacttgtgctaaattagcccatgtttcttttccttctgtaattatcgattctacttttttcggagttaaattgagttgttgagcgatttttcctattttgactgcttcagagtgattcgctaccaaagaatcaaatattgtcatgatttccaaatattttgcaagaactggcagttccctatttttgtctctgattgccaataaattttgagaggccgtacttcgacaagacctggaaaagcggaatattgtcaaccagttgttaacactttcccacccttccaggccagctagactagtttattttcctacaacttttgtaatctcccccttcacgtagtgaacaaattcttctaactgatctgctctagaagctaacctatcttcctcttgtcctaaataagcttgacgtgcttccaaaattacgggttgatattgagcaggtaatcgttccatggcccaatcggctgccacgtctttaggagcaattttccctgtaactgcagaataccaaatacgagataaagtaagtacaacatttcgttcatcacctgcccagtcggggggactattccataaggttaaagtttcatttaaagcttcgaataagtcttgctcagggacagggtcaaataactcttcggcagcagggccaactaaggccacgctgtgttctctggctttggtaagaagaatcgctaaatcaatatcaatagtggcaggttcaaaaattcctgctaaaatatcattcctttgccattctccaaattgtaactcacgcttcgctggatatctccagggaataatatcgtcatgtactactatggtcacttcaactgcccgaagaatctcagattccccaggggatgcacttgtttctaacaaatcattaattaaggcacgacgagttgtttcatccaggcgaacggtgacagttacaagtaaatctatgtcactgtgaggttttaatcctccatctacagcagatccatataaatggacagctaataaggtgggttctaaatgacgttcgattactcctacaacttcagagagttgtgtgctaacctcggcaataacggcttcacgcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtacaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagtggatccgaattcgagctccgtcgacaagcttgcggccgcactcgagcaccaccaccaccaccactgagatccggctgctaagcttctcacactggctcaccttcgggtgggcctttctgcgtttatatactagagagagaatataaaaagccagattattaatccggcttttttattatttctaggacagcttaaccggtacatcaaaatacccaaatccttactctcccctcaatatacaacaaaaaaaactttttgattcactttagtcataaaaattagaatttatctaccgaaatattacataaatgtaatgtatatattttctgatttattccgtgtgagccatgattcataatttataattcataatttctaaatatgcccctacaatggatatagaatgtcattttaattataggtatcataatcgtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaagg

SEQ ID NO: 13 - DNA, 8022 bp, Artificial Sequence
  18 copies of monomeric unit of MaSp1
ggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcttgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcctgatttggttgacgtagtttttgaatcgcttttgcctgaatttgtctaactctttcccttgataaattcataattgaggcagtatcactaagggaataaattttgccattttcaaacccataacgcagtctcagaattttttgttctctttcattgagtaaagaaagcatagagcgcaaatccttttgcatagatactctcattaagttttcttcgggagttgcagattctgtttcaattaattccactaattcagtgttataatcttctcccactttcatttctaaggatatggagcggggtatttgatttaaaaattgacgcaaatcaagggaagtcatatccatttctttggctatttcatctactgtggcgattctgcccaatttttgggataattgtctctgtactttttttagttgatttaatctttcagtaatatggataggaagacgaatggttctagcttgagttgcgatcgctcttgtcattccctgacgaatccaccaataagcataagtgctgaaatgatagcctttggtatgatcaaatttttcaacggctttttctaaaccgagagtaccttcttgaattaagtctaataattctaatcctctattttgatattttttggcaatagagacaactaaacgcaaattagcattaatcattttctgttttgctttaatgccattagccaaaatagtttctaactctgatagtttgacttgtgctaaattagcccatgtttcttttccttctgtaattatcgattctacttttttcggagttaaattgagttgttgagcgatttttcctattttgactgcttcagagtgattcgctaccaaagaatcaaatattgtcatgatttccaaatattttgcaagaactggcagttccctatttttgtctctgattgccaataaattttgagaggccgtacttcgacaagacctggaaaagcggaatattgtcaaccagttgttaacactttcccacccttccaggccagctagactagtttattttcctacaacttttgtaatctcccccttcacgtagtgaacaaattcttctaactgatctgctctagaagctaacctatcttcctcttgtcctaaataagcttgacgtgcttccaaaattacgggttgatattgagcaggtaatcgttccatggcccaatcggctgccacgtctttaggagcaattttccctgtaactgcagaataccaaatacgagataaagtaagtacaacatttcgttcatcacctgcccagtcggggggactattccataaggttaaagtttcatttaaagcttcgaataagtcttgctcagggacagggtcaaataactcttcggcagcagggccaactaaggccacgctgtgttctctggctttggtaagaagaatcgctaaatcaatatcaatagtggcaggttcaaaaattcctgctaaaatatcattcctttgccattctccaaattgtaactcacgcttcgctggatatctccagggaataatatcgtcatgtactactatggtcacttcaactgcccgaagaatctcagattccccaggggatgcacttgtttctaacaaatcattaattaaggcacgacgagttgtttcatccaggcgaacggtgacagttacaagtaaatctatgtcactgtgaggttttaatcctccatctacagcagatccatataaatggacagctaataaggtgggttctaaatgacgttcgattactcctacaacttcagagagttgtgtgctaacctcggcaataacggcttcacgcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtacaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagtggatccgaattcgagctccgtcgacaagcttgcggccgcactcgagcaccaccaccaccaccactgagatccggctgctaagcttctcacactggctcaccttcgggtgggcctttctgcgtttatatactagagagagaatataaaaagccagattattaatccggcttttttattatttctaggacagcttaaccggtacatcaaaatacccaaatccttactctcccctcaatatacaacaaaaaaaactttttgattcactttagtcataaaaattagaatttatctaccgaaatattacataaatgtaatgtatatattttctgatttattccgtgtgagccatgattcataatttataattcataatttctaaatatgcccctacaatggatatagaatgtcattttaattataggtatcataatcgtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaagg

SEQ ID NO: 14 - DNA, 9858 bp, Artificial Sequence
  36 copies of monomeric unit of MaSp1
ggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcttgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcctgatttggttgacgtagtttttgaatcgcttttgcctgaatttgtctaactctttcccttgataaattcataattgaggcagtatcactaagggaataaattttgccattttcaaacccataacgcagtctcagaattttttgttctctttcattgagtaaagaaagcatagagcgcaaatccttttgcatagatactctcattaagttttcttcgggagttgcagattctgtttcaattaattccactaattcagtgttataatcttctcccactttcatttctaaggatatggagcggggtatttgatttaaaaattgacgcaaatcaagggaagtcatatccatttctttggctatttcatctactgtggcgattctgcccaatttttgggataattgtctctgtactttttttagttgatttaatctttcagtaatatggataggaagacgaatggttctagcttgagttgcgatcgctcttgtcattccctgacgaatccaccaataagcataagtgctgaaatgatagcctttggtatgatcaaatttttcaacggctttttctaaaccgagagtaccttcttgaattaagtctaataattctaatcctctattttgatattttttggcaatagagacaactaaacgcaaattagcattaatcattttctgttttgctttaatgccattagccaaaatagtttctaactctgatagtttgacttgtgctaaattagcccatgtttcttttccttctgtaattatcgattctacttttttcggagttaaattgagttgttgagcgatttttcctattttgactgcttcagagtgattcgctaccaaagaatcaaatattgtcatgatttccaaatattttgcaagaactggcagttccctatttttgtctctgattgccaataaattttgagaggccgtacttcgacaagacctggaaaagcggaatattgtcaaccagttgttaacactttcccacccttccaggccagctagactagtttattttcctacaacttttgtaatctcccccttcacgtagtgaacaaattcttctaactgatctgctctagaagctaacctatcttcctcttgtcctaaataagcttgacgtgcttccaaaattacgggttgatattgagcaggtaatcgttccatggcccaatcggctgccacgtctttaggagcaattttccctgtaactgcagaataccaaatacgagataaagtaagtacaacatttcgttcatcacctgcccagtcggggggactattccataaggttaaagtttcatttaaagcttcgaataagtcttgctcagggacagggtcaaataactcttcggcagcagggccaactaaggccacgctgtgttctctggctttggtaagaagaatcgctaaatcaatatcaatagtggcaggttcaaaaattcctgctaaaatatcattcctttgccattctccaaattgtaactcacgcttcgctggatatctccagggaataatatcgtcatgtactactatggtcacttcaactgcccgaagaatctcagattccccaggggatgcacttgtttctaacaaatcattaattaaggcacgacgagttgtttcatccaggcgaacggtgacagttacaagtaaatctatgtcactgtgaggttttaatcctccatctacagcagatccatataaatggacagctaataaggtgggttctaaatgacgttcgattactcctacaacttcagagagttgtgtgctaacctcggcaataacggcttcacgcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtacaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagtggatccgaattcgagctccgtcgacaagcttgcggccgcactcgagcaccaccaccaccaccactgagatccggctgctaagcttctcacactggctcaccttcgggtgggcctttctgcgtttatatactagagagagaatataaaaagccagattattaatccggcttttttattatttctaggacagcttaaccggtacatcaaaatacccaaatccttactctcccctcaatatacaacaaaaaaaactttttgattcactttagtcataaaaattagaatttatctaccgaaatattacataaatgtaatgtatatattttctgatttattccgtgtgagccatgattcataatttataattcataatttctaaatatgcccctacaatggatatagaatgtcattttaattataggtatcataatcgtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaagg

SEQ ID NO: 15 - DNA, 6851 bp, Artificial Sequence
  2 copies of seq id no. 12
ttgaggacgaggttcataaatatccccttttttcttaaaataaatcttgtcatcagataaaagtaaatgggcggcataacagaaaaccggtgtttcctcctcaaaaatgagagttgctaattcagccggagtaacaggagttgcttcttcaatcaataactcccaagcaatttccaagctagaagcatctaaatgaggggttacttcttttacaaatcggggaatatcggtatatttaaaggattctccttctactaaaaaatctactttttggggtctaactttgtgggaattgccgttttcatctatggctatccagtcttttttcccttctggtttctcaatgacacctaagcggcgatcgccatttactttaaattcaactaatgttcctttttccacccgtgaatccttgttactaaattgactgagttggttattttatcgttactcttttaataatttaccatcaatatttcaattagcaattaacaattagcaatcaaaggtaaagggcaaggggcaaatccccctttatcccccctcgagaggggggagggcaaagatgaatagtgaataactgatgttaggcaaaaatagaatacatgattggttttaggtgtcaggtgtcgggttaaagagttgacaagaaatagatttgatatgatgtttattgattttcttctttattcttgtacaagagtctattgaagtatgggcgtacaagggcgttaatgagcgacatttgaattatttatgtaaaacctttgacatttggaatcaatgtttgagattcttaaacctgttacctcttacctgctacctgctacctgctacctgaaacctccctatttctattcactactccaaactttccaatatcttaaaactgtaccaaatgtactgcacaatgactgcggtgaataacataactactgacactgcctaataataattcgctcacggtagaatgtcctcttctacccataacaattagatttgctccccattcctgagcctgttgacaaataatagtacccggatgcccgtaaatttgattaaattctgcttttagcccttgatcttttgcctgatttgtccagtattgcaacatttcgatcgactcttgtttatatttttcccattgatgccgtacttcgacaagacctggaaaagcggaatattgtcaaccagttgttaacactttcccacccttccaggccagctagactagtctaagtagctgtagaaggatctatatcaaaatgcattacttcttctctgatacctagtttcgtataaagtgcaacggcagggtcatcaccgtagtcagcctgcacataaattacatacgcccctaatgcatttgcttcatgtttcaataaattgattaaggctgtggcaattccttgacgtctatgttccccagaaactgctaaatcatagatataaatttcagaacgaggctgctcaaatttaggtaaaacatatgcagctaacgcaccaacgactgcctcctgatcaaacgcagccaaagcaataaacgttttgcttctaagtaaatttcctagatagtcactatcgggttgatgctgagaataggttgccacatccccaaactcgcgtccaaataaatctaaggctgcacgcattgatttcacttggtcaggaccaagtctacaagtcctaataatacccatagaagatccccccagttttgttttaggccttgatccttgttgggtcacatcattagaactccttagcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtacaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagccttatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagtggatccgaattcgagctccgtcgacaagcttgcggccgcactcgagcaccaccaccaccaccactgagatccggctgctaagcttctcacactggctcaccttcgggtgggcctttctgcgtttatatactagagagagaatataaaaagccagattattaatccggcttttttattatttctaggacagcttaaccctattgtatttttctttttctcctattcctagatcttgtaatgaatcattactctctgaaatatagctactaattttatggttgtttgtaaaatatattaacaaatgaacaataaatcatattttgtgttaatctaattattagacaactactgaatttatattcagatattcacagataggagaattttgattatgactaccactacagtaactcaaatgaaatgtgcttgtccctcttgtttatgtataattgatattagtcaagcaatttccagagatgggcattattattgctcaacggcttgtgccgaaggacataaggaaggagaaggatgtggccatagcggttgtggatgccattcttagttataaaaggcgaagacaaattaggaattgttgaaaaagtggtgtcaggtgttggggatagcaagacgagggaggaacaagtaataagtaacctcagttcggtttaagaatatccgataaggttaggtgtcaggttgcaggtgttagggagataaggggagagggtgagagggtgaagtaggggttgaacatatttaacgtcagttcgggttagggcaattttatcgttatttctgagaagaagctataaggttttctgattacgagttggtctgctttcagcttatccaaaactcaggttaagtaatcagtaatgagtgttaggagtttttaattctcactcttttttcaggtttgcaataaattcttggtgactaattgcaaattataaatttattattgtgttgagaatgagtaggtaagttataatgagttgtttggatgtatataattgttaaccaaaaaaaatttagaaatacttaaagaatgatttggccttttaaaccttcgactcgtaaacaaattgctcgaattgaaatcaatggtgcgatcgcatctgccactcgtgagagagtattagaagcattggaagaagtagaagagagggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcttaatttcttctacctgagt

SEQ ID NO: 16 - DNA, 214 bp, Artificial Sequence
  promoter
ttaccaattggtggcggccttaaccccctaagccctatgtctgacacccagtcaccccaaccgccttagtcggtattccccactgggtgggcgatacacgatttcccggaaaaattgaaaaaaaattttgtggcaggtgatatgatttgcgatcgaggtgggtaagttagaactacagaaaacaaataacctcatttcaccccgaggagaacga

SEQ ID NO: 17 - DNA, 35 bp, Artificial Sequence
  promoter
ttgacagctagctcagtcctaggtataatgctagc

SEQ ID NO: 18 - DNA, 45 bp, Artificial Sequence
  promoter
gagtattgacttcgcatctttttgtacctataatagattcatctg

SEQ ID NO: 19 - DNA, 219 bp, Artificial Sequence
  promoter
tattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcg

SEQ ID NO: 20 - DNA, 281 bp, Artificial Sequence
  promoter
acaattaataacttcttcctgtacgggcgaatggccatttgctcctaactaactccgtactgctttgcggaacgagcgtagcgaactctccgaattactaagccttcatccctgatagatgcaaaaaacgaattaaaattatgtgtaaaaagaaaatgtgtctttatttagtagtcaaagttacaaaatattaagaatcaaattaataatgtattgggcagttaagtatataagtctttaaatatttatttgtattcaatatattaaggaggatcagcctt

SEQ ID NO: 21 - DNA, 421 bp, Artificial Sequence
  promoter
caactttagatattcgtagttggcaatgtcgtaaatgcggaacaatacatggaaaacatatagatttgtaatgagaaaaagtgtaaacaaatattaagaaaaagatcagaaaaatttaacaacacgtaataaaaaaatgcgtcactacgggttataaatttacatgaaaggttaaaacacttttctgagacgattttgataaaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattccacgtttttactttgaaagtaaaggagaatatgagatt

SEQ ID NO: 22 - DNA, 2508 bp, Artificial Sequence
  locus on algae
ccttcttccgttaataaggattcaatcaaaggattaacccattgtgccgccatagcacaaaattcttcattatcgttaacgcattccatctggacataattgacatcgggatatttaacgtataatgcttgaataatgtgttctacatctaacaaggtttcatggttttccgttgcaaagccaatgggcataaatattaaggcttttgcccctaactcaatcaagtttcttcctgctaactccgcattaggttgagtccatttaattagaggggtttgatggttgagccaacctacggaaattaagggataacgataaatgagtttttctcttaccaattcatataaggcttgactttcatcaatacccgaagtaaatccttttgcttcatgagggcaaccgtgattcattaaaacaataccaatttctgaaggtaaataagcattgcttaattttgcgtcgatttcttcttctactaatttagctaataaatcgatatactctggtttattgtagaaagaaggtatataacgtaaggctttaacccaatggtcttcttttttgtctttttgtgctaacgcctcgtttatttgctcgatcgcaattccactggtgaagatagaatcaactactaagaggggataaatgagaattttctcaaatccttcagctttaacctgctctaaaacttgatagggtaaatgaggtttacagaagttgaaagccttaaatactttgactttttctccccaagtgtgttgcaactgatgctcaatttttgctctttgacgctcgaaaatagcattatgaggagaaacaaaattgccgtgttgatgactccattcatgtaaatcgaaaaccgctaacaatttagcaagggggggatataaccatgttggcacaggggcaaattttgccgtaagtaggtttaaggcttgttcattatagttggcaaaatcatcataactttccacctctccatatcccataagaataacggctactctatctgtagcaggtgagactgaatttgattcaatagttttttccggagtaactaccacgattatgatacctataattaaaatgacattctatatccattgtaggggcatatttagaaattatgaattataaattatgaatcatggctcacacggaataaatcagaaaatatatacattacatttatgtaatatttcggtagataaattctaatttttatgactaaagtgaatcaaaaagttttttttgttgtatattgaggggagagtaaggatttgggtattttgatgtacctaaaacaactcaaaattaaatagtagttatcaaacagggacagagattatgaccaactttgatgttaaacctcttaaagaccgtccttccacctccccgacatcttcatcttcgggtcataattcaagggcgacatactctccaccgtcaacccaaaatagtccctacataaattctcgtgatttgatgcgcctgtatcttcgtgatattgggcgcattcctttattaaccaaagaagaagaaattactttagctaaagaaattaaagcctctcaaaatttattggcaatcagagacaaaaatagggaactgccagttcttgcaaaatatttggaaatcatgacaatatttgattctttggtagcgaatcactctgaagcagtcaaaataggaaaaatcgctcaacaactcaatttaactccgaaaaaagtagaatcgataattacagaaggaaaagaaacatgggctaatttagcacaagtcaaactatcagagttagaaactattttggctaatggcattaaagcaaaacagaaaatgattaatgctaatttgcgtttagttgtctctattgccaaaaaatatcaaaatagaggattagaattattagacttaattcaagaaggtactctcggtttagaaaaagccgttgaaaaatttgatcataccaaaggctatcatttcagcacttatgcttattggtggattcgtcagggaatgacaagagcgatcgcaactcaagctagaaccattcgtcttcctatccatattactgaaagattaaatcaactaaaaaaagtacagagacaattatcccaaaaattgggcagaatcgccacagtagatgaaatagccaaagaaatggatatgacttcccttgatttgcgtcaatttttaaatcaaataccccgctccatatccttagaaatgaaagtgggagaagattataacactgaattagtggaattaattgaaacagaatctgcaactcccgaagaaaacttaatgagagtatctatgcaaaaggatttgcgctctatgctttctttactcaatgaaagagaacaaaaaattctgagactgcgttatgggtttgaaaatggcaaaatttattcccttagtgatactgcctcaattatgaatttatcaagggaaagagttagacaaattcaggcaaaagcgattcaaaaactacgtcaaccaaatcag

SEQ ID NO: 23 - DNA, 2403 bp, Artificial Sequence
  locus on algae
ttaatttcttctacctgagtttgaggacgaggttcataaatatccccttttttcttaaaataaatcttgtcatcagataaaagtaaatgggcggcataacagaaaaccggtgtttcctcctcaaaaatgagagttgctaattcagccggagtaacaggagttgcttcttcaatcaataactcccaagcaatttccaagctagaagcatctaaatgaggggttacttcttttacaaatcggggaatatcggtatatttaaaggattctccttctactaaaaaatctactttttggggtctaactttgtgggaattgccgttttcatctatggctatccagtcttttttcccttctggtttctcaatgacacctaagcggcgatcgccatttactttaaattcaactaatgttcctttttccacccgtgaatccttgttactaaattgactgagttggttattttatcgttactcttttaataatttaccatcaatatttcaattagcaattaacaattagcaatcaaaggtaaagggcaaggggcaaatccccctttatcccccctcgagaggggggagggcaaagatgaatagtgaataactgatgttaggcaaaaatagaatacatgattggttttaggtgtcaggtgtcgggttaaagagttgacaagaaatagatttgatatgatgtttattgattttcttctttattcttgtacaagagtctattgaagtatgggcgtacaagggcgttaatgagcgacatttgaattatttatgtaaaacctttgacatttggaatcaatgtttgagattcttaaacctgttacctcttacctgctacctgctacctgctacctgaaacctccctatttctattcactactccaaactttccaatatcttaaaactgtaccaaatgtactgcacaatgactgcggtgaataacataactactgacactgcctaataataattcgctcacggtagaatgtcctcttctacccataacaattagatttgctccccattcctgagcctgttgacaaataatagtacccggatgcccgtaaatttgattaaattctgcttttagcccttgatcttttgcctgatttgtccagtattgcaacatttcgatcgactcttgtttatatttttcccattgatgcactcttgtttatatttttcccattgatgctgtaattcttttattatttccgtgtcgtaagtaaaagagtaaggagcaaaactaaggggagtttctgtcgtttcctgagacaacacatgaagaaaattgagttgagagtgacaatttttccctaaatacaaagcagtttcaaagacactttgtgctatgggggagcgatcgagggataccaatatcttgtcatacatacttatttgcctcactattagccctatatgtctctattgtatttttctttttctcctattcctagatcttgtaatgaatcattactctctgaaatatagctactaattttatggttgtttgtaaaatatattaacaaatgaacaataaatcatattttgtgttaatctaattattagacaactactgaatttatattcagatattcacagataggagaattttgattatgactaccactacagtaactcaaatgaaatgtgcttgtccctcttgtttatgtataattgatattagtcaagcaatttccagagatgggcattattattgctcaacggcttgtgccgaaggacataaggaaggagaaggatgtggccatagcggttgtggatgccattcttagttataaaaggcgaagacaaattaggaattgttgaaaaagtggtgtcaggtgttggggatagcaagacgagggaggaacaagtaataagtaacctcagttcggtttaagaatatccgataaggttaggtgtcaggttgcaggtgttagggagataaggggagagggtgagagggtgaagtaggggttgaacatatttaacgtcagttcgggttagggcaattttatcgttatttctgagaagaagctataaggttttctgattacgagttggtctgctttcagcttatccaaaactcaggttaagtaatcagtaatgagtgttaggagtttttaattctcactcttttttcaggtttgcaataaattcttggtgactaattgcaaattataaatttattattgtgttgagaatgagtaggtaagttataatgagttgtttggatgtatataattgttaaccaaaaaaaatttagaaatacttaaagaatgatttggccttttaaaccttcgactcgtaaacaaattgctcgaattgaaatcaatggtgcgatcgcatctgccactcgtgagagagtattagaagcattggaagaagtagaagagag

SEQ ID NO: 24 - DNA, 2403 bp, Artificial Sequence
  locus on algae
cacggaaattattgttgatagctctttaggtaatcaaaacaatcctgatgtggatctcgctcctgatggtagtatcctcattagttataccgatgaaagttctaacagtgtgaaatatcgtcagtttaacagtaacggtgaacctctaggagatagtgccacttttgacgttgagggcaatcaagaaactaactctgccatagcagtaggaactaataataaagcagtagttgtcgctgatgcaggaaataacaatagttttaacccttatggtcaaattctaactcctgatgctagtaatactctcaatatccctttaaatcgctttcaaaataccagtcaacccggcacatatttatttgcaggggagcaagaaagtcaaaatattcgtcagaatttccccaactttgtggaagaaggatttgctttttctgtggcggaaaaccctcaagataacttaattcgttttaaccgttttcaaaacagcgatcgccccggtacatatttatttgcaggggagcaagaaagtcaaaatattcgtcagaatttccccaactttatcgaagaaggagtcgctttttatgcctttggtgcaggttcaaatcaagccactcctttttatcgtttccaaaatactgatgtacccggtacttacctttttgttggtgatagtgaaagacaaagtattcttcagaatttccccaattttgtagaagagggcattgcttttgaagcggctacttagttagcttaatcggcgagaagccccacgcttagttttcgacaaaaaaattaaacaagcgtgggacggggcttttaaagagcggaaatagttccgctcactgcccctcatgaatcgccagtgacgtgttataatgactaaaaagtggggctggacgtgtacctcactaaaaaacgccgtagtgggaaagaaggagtaccacgatggaagttcaagaaggataatttccgcttgaagcctacactctgcgtaagcaagtgtatggagcgtcactattcgccatcaacgagaatcaattgatttaatcatggttgtaggggaaaaactaccccagacaacatcaaacaaatgctcaacaaatgttattatcttttacataaattaagaagataaaaactggagaataaaaaaatggactggagagtgttaatcgtattattaccccttggagttgccgcaggttgggcattatttaatattggtgctgctgcgatcgcacaattccaaaaagttctcaacaaacaaaattcttaacttcagaggctaaaacgcaagggggtaaaaccccttttttaactgagttcggaataaattttcatctatgaataatggcgaaaagggcaagaagcaaaggcaaaccctaacacctgaaacctgaaacctgaaacctacccttacccaatattcttaaaccgaactgaggttaaataagtcttcctcttttcaatccttaataaattttaacgatcgccgcttccatattgcaacaattccaaataacgatgatagtaatactgttgtcgaggagaaaatttttcagagaaaccaataaaacccttagcgaaaggatataaacccgtgtaaactccaagactaagataatgtttgagccaatcagctaaaatgggtaaacccacttgaggaagtaaaggtaaaactaacatcggattaactagcggtaaagtctttgttaagcccgaaaactgcaccacatcttgtaaaaaaggctttagcacatcgtcacctaatttatccattacccgaaaaacaccgctcattaaatcgttgatttgattaggttggggttgcttatcaataggcacactcatagttttttgaaataaccaagtaacagaaatatttggttgataaggctgaattaaagctaaatcctcacgggttaaataatctccttttaaggcttcatcaatggctaaacttaaacgaggtaaatgacgcatcatcgccccaaaaccaccaaaactgacaggagattgcataccactactatcccccaccgctataattctatcccaaggcatttttaaaggactttgacggtaagcggggaaaaaaccaaataaaaaccgttgaaaatcgagattatctaattctatttgttgatattcgggcagtaatcttaaatattccgtcattaagttttttaggctaattctttcagggttagcatccacataggtaaaaagataggttgtgcgtccatccttagctggaaaagcctcccagaaatattgacattgattaataatcggagtaaaagaataaattaaatctcctgtttcattctgtttaaatccttttccacaactaccgacaactaaacaaactccatcgggtttttctccgtttcttatctgttt

SEQ ID NO: 25 - DNA, 397 bp, Artificial Sequence
  promoter
caactttagatattcgtagttggcaatgtcgtaaatgcggaacaatacatggaaaacatatagatttgtaatgagaaaaagtgtaaacaaatattaagaaaaagatcagaaaaatttaacaacacgtaataaaaaaatgcgtcactacgggttataaatttacatgaaaggttaaaacacttttctgagacgattttgataaaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattaggagattaactaca

SEQ ID NO: 26 - DNA, 7346 bp, Artificial Sequence
  10 copies of monomeric unit of MaSp1
ggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcttgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcctgatttggttgacgtagtttttgaatcgcttttgcctgaatttgtctaactctttcccttgataaattcataattgaggcagtatcactaagggaataaattttgccattttcaaacccataacgcagtctcagaattttttgttctctttcattgagtaaagaaagcatagagcgcaaatccttttgcatagatactctcattaagttttcttcgggagttgcagattctgtttcaattaattccactaattcagtgttataatcttctcccactttcatttctaaggatatggagcggggtatttgatttaaaaattgacgcaaatcaagggaagtcatatccatttctttggctatttcatctactgtggcgattctgcccaatttttgggataattgtctctgtactttttttagttgatttaatctttcagtaatatggataggaagacgaatggttctagcttgagttgcgatcgctcttgtcattccctgacgaatccaccaataagcataagtgctgaaatgatagcctttggtatgatcaaatttttcaacggctttttctaaaccgagagtaccttcttgaattaagtctaataattctaatcctctattttgatattttttggcaatagagacaactaaacgcaaattagcattaatcattttctgttttgctttaatgccattagccaaaatagtttctaactctgatagtttgacttgtgctaaattagcccatgtttcttttccttctgtaattatcgattctacttttttcggagttaaattgagttgttgagcgatttttcctattttgactgcttcagagtgattcgctaccaaagaatcaaatattgtcatgatttccaaatattttgcaagaactggcagttccctatttttgtctctgattgccaataaattttgagaggccgtacttcgacaagacctggaaaagcggaatattgtcaaccagttgttaacactttcccacccttccaggccagctagactagtttattttcctacaacttttgtaatctcccccttcacgtagtgaacaaattcttctaactgatctgctctagaagctaacctatcttcctcttgtcctaaataagcttgacgtgcttccaaaattacgggttgatattgagcaggtaatcgttccatggcccaatcggctgccacgtctttaggagcaattttccctgtaactgcagaataccaaatacgagataaagtaagtacaacatttcgttcatcacctgcccagtcggggggactattccataaggttaaagtttcatttaaagcttcgaataagtcttgctcagggacagggtcaaataactcttcggcagcagggccaactaaggccacgctgtgttctctggctttggtaagaagaatcgctaaatcaatatcaatagtggcaggttcaaaaattcctgctaaaatatcattcctttgccattctccaaattgtaactcacgcttcgctggatatctccagggaataatatcgtcatgtactactatggtcacttcaactgcccgaagaatctcagattccccaggggatgcacttgtttctaacaaatcattaattaaggcacgacgagttgtttcatccaggcgaacggtgacagttacaagtaaatctatgtcactgtgaggttttaatcctccatctacagcagatccatataaatggacagctaataaggtgggttctaaatgacgttcgattactcctacaacttcagagagttgtgtgctaacctcggcaataacggcttcacgcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtcaactttagatattcgtagttggcaatgtcgtaaatgcggaacaatacatggaaaacatatagatttgtaatgagaaaaagtgtaaacaaatattaagaaaaagatcagaaaaatttaacaacacgtaataaaaaaatgcgtcactacgggttataaatttacatgaaaggttaaaacacttttctgagacgattttgataaaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattccacgtttttactttgaaagtaaaggagaatatgagattatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagtggatccgaattcgagctccgtcgacaagcttgcggccgcactcgagcaccaccaccaccaccactgagatccggctgctaagcttctcacactggctcaccttcgggtgggcctttctgcgtttatatactagagagagaatataaaaagccagattattaatccggcttttttattatttctaggacagcttaaccggtacatcaaaatacccaaatccttactctcccctcaatatacaacaaaaaaaactttttgattcactttagtcataaaaattagaatttatctaccgaaatattacataaatgtaatgtatatattttctgatttattccgtgtgagccatgattcataatttataattcataatttctaaatatgcccctacaatggatatagaatgtcattttaattataggtatcataatcgtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaagg

SEQ ID NO: 27 - DNA, 7322 bp, Artificial Sequence
  10 copies of monomeric unit of MaSp1
ggcgcgccacctgcaggcatgcaagcttggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtcttgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggaaattcgagctcaaggcccaggcggccgcgtatgtgaattcctgatttggttgacgtagtttttgaatcgcttttgcctgaatttgtctaactctttcccttgataaattcataattgaggcagtatcactaagggaataaattttgccattttcaaacccataacgcagtctcagaattttttgttctctttcattgagtaaagaaagcatagagcgcaaatccttttgcatagatactctcattaagttttcttcgggagttgcagattctgtttcaattaattccactaattcagtgttataatcttctcccactttcatttctaaggatatggagcggggtatttgatttaaaaattgacgcaaatcaagggaagtcatatccatttctttggctatttcatctactgtggcgattctgcccaatttttgggataattgtctctgtactttttttagttgatttaatctttcagtaatatggataggaagacgaatggttctagcttgagttgcgatcgctcttgtcattccctgacgaatccaccaataagcataagtgctgaaatgatagcctttggtatgatcaaatttttcaacggctttttctaaaccgagagtaccttcttgaattaagtctaataattctaatcctctattttgatattttttggcaatagagacaactaaacgcaaattagcattaatcattttctgttttgctttaatgccattagccaaaatagtttctaactctgatagtttgacttgtgctaaattagcccatgtttcttttccttctgtaattatcgattctacttttttcggagttaaattgagttgttgagcgatttttcctattttgactgcttcagagtgattcgctaccaaagaatcaaatattgtcatgatttccaaatattttgcaagaactggcagttccctatttttgtctctgattgccaataaattttgagaggccgtacttcgacaagacctggaaaagcggaatattgtcaaccagttgttaacactttcccacccttccaggccagctagactagtttattttcctacaacttttgtaatctcccccttcacgtagtgaacaaattcttctaactgatctgctctagaagctaacctatcttcctcttgtcctaaataagcttgacgtgcttccaaaattacgggttgatattgagcaggtaatcgttccatggcccaatcggctgccacgtctttaggagcaattttccctgtaactgcagaataccaaatacgagataaagtaagtacaacatttcgttcatcacctgcccagtcggggggactattccataaggttaaagtttcatttaaagcttcgaataagtcttgctcagggacagggtcaaataactcttcggcagcagggccaactaaggccacgctgtgttctctggctttggtaagaagaatcgctaaatcaatatcaatagtggcaggttcaaaaattcctgctaaaatatcattcctttgccattctccaaattgtaactcacgcttcgctggatatctccagggaataatatcgtcatgtactactatggtcacttcaactgcccgaagaatctcagattccccaggggatgcacttgtttctaacaaatcattaattaaggcacgacgagttgtttcatccaggcgaacggtgacagttacaagtaaatctatgtcactgtgaggttttaatcctccatctacagcagatccatataaatggacagctaataaggtgggttctaaatgacgttcgattactcctacaacttcagagagttgtgtgctaacctcggcaataacggcttcacgcattattttctccttttttcgggttaatcctccatctaaggttgtttaatttctttggctagaaacacccgatgccttagatgtgcgaattttgacaacttttctagtatatcattgtcttttttttttttccagatttaccccttgttttattttcttgatggaagttaagatctttttagggtcatctcatttaacagttagttatagggaaaattggcgaggtcaactttagatattcgtagttggcaatgtcgtaaatgcggaacaatacatggaaaacatatagatttgtaatgagaaaaagtgtaaacaaatattaagaaaaagatcagaaaaatttaacaacacgtaataaaaaaatgcgtcactacgggttataaatttacatgaaaggttaaaacacttttctgagacgattttgataaaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattaggagattaactacaatgcaccatcatcatcatcattcttctggtctggtgccacgcggttctggtatgaaagaaaccgctgctgctaaattcgaacgccagcacatggacagcccagatctgggtaccgacgacgacgacaaggccatggctgatatcggatccatggctagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagcagcgggcgtggagggctgggggggcaaggtgcaggagcagccgcagccgctggaggtgccggacaggggggttatgggggattaggtagccagggtactagtggatccgaattcgagctccgtcgacaagcttgcggccgcactcgagcaccaccaccaccaccactgagatccggctgctaagcttctcacactggctcaccttcgggtgggcctttctgcgtttatatactagagagagaatataaaaagccagattattaatccggcttttttattatttctaggacagcttaaccggtacatcaaaatacccaaatccttactctcccctcaatatacaacaaaaaaaactttttgattcactttagtcataaaaattagaatttatctaccgaaatattacataaatgtaatgtatatattttctgatttattccgtgtgagccatgattcataatttataattcataatttctaaatatgcccctacaatggatatagaatgtcattttaattataggtatcataatcgtggtagttactccggaaaaaactattgaatcaaattcagtctcacctgctacagatagagtagccgttattcttatgggatatggagaggtggaaagttatgatgattttgccaactataatgaacaagccttaaacctacttacggcaaaatttgcccctgtgccaacatggttatatcccccccttgctaaattgttagcggttttcgatttacatgaatggagtcatcaacacggcaattttgtttctcctcataatgctattttcgagcgtcaaagagcaaaaattgagcatcagttgcaacacacttggggagaaaaagtcaaagtatttaaggctttcaacttctgtaaacctcatttaccctatcaagttttagagcaggttaaagctgaaggatttgagaaaattctcatttatcccctcttagtagttgattctatcttcaccagtggaattgcgatcgagcaaataaacgaggcgttagcacaaaaagacaaaaaagaagaccattgggttaaagccttacgttatataccttctttctacaataaaccagagtatatcgatttattagctaaattagtagaagaagaaatcgacgcaaaattaagcaatgcttatttaccttcagaaattggtattgttttaatgaatcacggttgccctcatgaagcaaaaggatttacttcgggtattgatgaaagtcaagccttatatgaattggtaagagaaaaactcatttatcgttatcccttaatttccgtaggttggctcaaccatcaaacccctctaattaaatggactcaacctaatgcggagttagcaggaagaaacttgattgagttaggggcaaaagccttaatatttatgcccattggctttgcaacggaaaaccatgaaaccttgttagatgtagaacacattattcaagcattatacgttaaatatcccgatgtcaattatgtccagatggaatgcgttaacgataatgaagaattttgtgctatggcggcacaatgggttaatcctttgattgaatccttattaacggaagaagg